SEQUENCE LISTING (45 sequences)

SEQ ID NO: 1 - DNA, 30 bp, Artificial Sequence
  Synthetic sequence
tggcgcgccgctctagcagaaggcatgttg

SEQ ID NO: 2 - DNA, 32 bp, Artificial Sequence
  Synthetic sequence
tcgcccttgctcaccatgatgcaccccgccta

SEQ ID NO: 3 - DNA, 33 bp, Artificial Sequence
  Synthetic sequence
gtaggcggggtgcatcatggtgagcaagggcga

SEQ ID NO: 4 - DNA, 26 bp, Artificial Sequence
  Synthetic sequence
tgaatagtgcatatcagcatacctta

SEQ ID NO: 5 - DNA, 36 bp, Artificial Sequence
  Synthetic sequence
tcgcccttgctcaccatctacggtcctggatgatcc

SEQ ID NO: 6 - DNA, 28 bp, Artificial Sequence
  Synthetic sequence
caatatacgctttatcaaataccatcac

SEQ ID NO: 7 - DNA, 26 bp, Artificial Sequence
  Synthetic sequence
cttcttcttcttcttttctcattgtc

SEQ ID NO: 8 - DNA, 25 bp, Artificial Sequence
  Synthetic sequence
aactcgagcaaataccatcacatcg

SEQ ID NO: 9 - DNA, 33 bp, Artificial Sequence
  Synthetic sequence
ggctcgagtttggattgagagtgaatatgagac

SEQ ID NO: 10 - DNA, 30 bp, Artificial Sequence
  Synthetic sequence
gatctagaatgtatggtttgcggaataaac

SEQ ID NO: 11 - DNA, 33 bp, Artificial Sequence
  Synthetic sequence
ccgagctctcaaccgatataatcaaggtcaaac

SEQ ID NO: 12 - DNA, 30 bp, Artificial Sequence
  Synthetic sequence
ggtctagaatggattctcaattggtcaatc

SEQ ID NO: 13 - DNA, 28 bp, Artificial Sequence
  Synthetic sequence
gcgagctcttattgcaacgatttgggct

SEQ ID NO: 14 - DNA, 28 bp, Artificial Sequence
  Synthetic sequence
cacccgagttattgcaacgatttgggct

SEQ ID NO: 15 - DNA, 26 bp, Artificial Sequence
  Synthetic sequence
ggtctagagttgttgtgactccgagg

SEQ ID NO: 16 - DNA, 19 bp, Artificial Sequence
  Synthetic sequence
ggaaacagctatgaccatg

SEQ ID NO: 17 - DNA, 62 bp, Artificial Sequence
  Synthetic sequence
tatgagctctcatgcgtaatctggcacatcataaggataaccgatataatcaaggtcaaacg

SEQ ID NO: 18 - DNA, 57 bp, Artificial Sequence
  Synthetic sequence
tatgagctcttatgcgtaatctggcacatcataaggatattgcaacgatttgggctg

SEQ ID NO: 19 - DNA, 33 bp, Artificial Sequence; identical to SEQ ID NO: 9
  Synthetic sequence

SEQ ID NO: 20 - DNA, 30 bp, Artificial Sequence
  Synthetic sequence
acggcgcgcccatgagcggagaattaaggg

SEQ ID NO: 21 - DNA, 29 bp, Artificial Sequence
  Synthetic sequence
cgtctagaatgtacccgtctaggaataaa

SEQ ID NO: 22 - DNA, 33 bp, Artificial Sequence
  Synthetic sequence
aggagctcttaaccaatatagtcaaggtcaaac

SEQ ID NO: 23 - DNA, 33 bp, Artificial Sequence
  Synthetic sequence
ggtgtacaatggattctcagttagtaaatcctc

SEQ ID NO: 24 - DNA, 34 bp, Artificial Sequence
  Synthetic sequence
gcggtaccttatttcaatgatttggcttgagaag

SEQ ID NO: 25 - DNA, 33 bp, Artificial Sequence
  Synthetic sequence
gccccgagttatttcaatgatttgcttgagaag

SEQ ID NO: 26 - DNA, 26 bp, Artificial Sequence
  Synthetic sequence
ggtctagagttgttgtgactccgagg

SEQ ID NO: 27 - DNA, 19 bp, Artificial Sequence
  Synthetic sequence
ggaaacagctatgaccatg

SEQ ID NO: 28 - DNA, 2959 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgagtatttttacaacaattaccaacaacaacaaacaacaaacaacattacaattactatttacaatctagaacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgagaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaatt

SEQ ID NO: 29 - DNA, 2538 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgagtatttttacaacaattaccaacaacaacaaacaacaaacaacattacaattactatttacaatctagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 30 - DNA, 2474 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 31 - DNA, 2782 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgagggaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctccccaacctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 32 - DNA, 2474 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataagagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 33 - DNA, 3024 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctctctcaacaatctagctagagtttgctcctatctatatgtaataaggtatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 34 - DNA, 3024 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatggattctcaattggtcaatcctcccaacgcatttaactacatagagtcccatcgggacgaatatcaactttctcatgacctcactgagataattctgcaattcccttccacggcgtcgcagttaacagctaggctgagccgtagctgcatgaagatcgaccactgcgtcatagagtacaggcaacaagtgccgattaacgcgacagggtcggtcattgtcgagatccacgacaagcgaatgacagacaacgaatcgctgcaggcttcctggacatttccaatcagatgcaacatagatctacattatttctcggcttcgttcttctcgttgaaagacccaattccatggaaactctattatagggtttgcgatacgaatgttcatcagaggacccacttcgccaagttcaaagggaaactgaaactatcgacggcgaaacactccgtggatatacccttccgggcaccgacagtaaagatcctgtcgaaacagttcacagataaagatgtcgacttttcacatgtcgactacggaagatgggaaaggaaacccataagatgcgcatccatgtcaaggattggactaccaggcccaatagagatcaagccgggtgaatcatgggcttcacggagtacaattgggctcagcaactccgatgccgactcagaggtggaaaacgcaatgcatccatataggcatctcagcaggcttggaacaagcgtcttagacccaggggagtctgcctcaattgtaggggcccagagagcggaatccaacattactatgtcgatggggcaattaaacgagctagtgaaaaacactgtccaggagtgtataaatagtaactgtaaggcttatcagcccaaatcgttgcaataagagctctctcaacaatctagctagagtttgctcctatctatatgtaataaggtatgctgatatgcactattcaaataggagcattagctatgtttgttaatgtcactttatgttatgtgggtaagtcacctaagacactccacgtacctacgttgttgtctcttaccggctttaataaatcttctgcccttgttccatatttactaattatccctttcttcactaaaagaaaattgttatcattaagtattagtctttagaacatatgaggtctttaattgggtaggttttacaaattaactaatataaaatgtcataaaatccacgtggttaaacaaatgcagaaaatcgacgtcgtctattggaccgacagttgctattaatataatgggccaccatagtagactgacaaataaattacctgacaacatcgtttcactaaataacaaacacaaaaagggagtgcattttccagggcatttttgtaataaaaaacagttaaaagggagtgcaatagaaatataggggtgtggaaatagtgatttgagcacgtcttgaagcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgactcaaccgatataatcaaggtcaaacgatacaaaagtagatgcctttgacatagtatctgacatccaacagtaatatactaggagggcgttcttgctgatgttgtcataaacacccttgcatgactcatggtcaagatccttaaaagtggaccaacaattatatcgcctgttagagagcgatgtggatccttccacgtcaaccatcatcgtatccttctccacggacaatacacgtttgaacacgtgtcttatgtagaatcggtctttcaaagaaggtgttatgctgagattaccatggctatggatccttgcaccgaacagctcgtcgaatgtatgcaggcatccagacgcacccaagtggggtttacgatccacaacaaccaccagagagaacactccttccacttttggggcagaaccgtccatgttcatatctggttgaacacgttcaatcttgacagtccctttgaaacgaagtcgtttcaacttaatataggacctcgatcggttgggttccatcttgcccttgctggggtagctgataaacgtagaaatggctgaattatgggccattacaaattcaggcccatactggttctcatgtatacgttgggctgacatcttgggctcgtcaatgggcttactaggattcatagctcgacgtttgccatcatgacgcttattagcggacaagcgatttaaaaaactgctacgtgaataaaatcggcgttggctgaacgatgaaccacgtttattccgcaaaccatacattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 35 - DNA, 2949 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 36 - DNA, 2824 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgatagccatgctagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 37 - DNA, 3039 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgaaattcgcttcaagacgtgctcaaatcactatttccacacccctatatttctattgcactcccttttaactgttttttattacaaaaatgccctggaaaatgcactccctttttgtgtttgttatttagtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccggtaagagacaacaacgtaggtacgtggagtgtcttaggtgacttacccacataacataaagtgacattaacaaacatagctaatgctcctatttgaatagtgcatatcagcataccttattacatatagataggagcaaactctagctagattgttgagagagctcttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggtttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 38 - DNA, 2783 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctccccaacctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 39 - DNA, 2308 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgattaatgataattgtaataaaaagctattattgaactttcaattcctcaacaaagaaattattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggtttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 40 - DNA, 2534 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagattgtaaatagtaattgtaatgttgtttgttgtttgttgttgttggtaattgttgtaaaaatactcgatcgagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 41 - DNA, 2853 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgagggaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctccccaacctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagttattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggcttcctttcccatcttccgtagtcgacatgtgaaaagtcgacatctttatctgtgaactgtttcgacaggatctttactgtcggtgcccggaagggtatatccacggagtgtttcgccgtcgatagtttcagtttccctttgaacttggcgaagtgggtcctctgatgaacattcgtatcgcaaaccctataatagagtttccatggaattgggtctttcaacgagaagaacgaagccgagaaataatgtagatctatgttgcatctgattggaaatgtccaggaagcctgcagcgattcgttgtctgtcattcgcttgtcgtggatctcgacaatgaccgaccctgtcgcgttaatcggcacttgttgcctgtactctatgacgcagtggtcgatcttcatgcagctacggctcagcctagctgttaactgcgacgccgtggaagggaattgcagaattatctcagtgaggtcatgagaaagttgatattcgtcccgatgggactctatgtagttaaatgcgttgggaggattgaccaattgagaatccattctagattgtaaatagtaattgtaatgttgtttgttgtttgttgttgttggtaattgttgtaaaaatactcgatcgagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 42 - DNA, 2477 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtacccgtctaggaataaacgtggatcctactttaatcaacgtcgacagtattcacgcaatcatgtgtggaaacggccaactgctgcgaagagacatgactggaagcgtcgaccttcaaatacgagcaagcccaacgacgagcccaagatgtcagcccaacgcatacatgagaatcagtatgggcctgagtttgtaatggcccagaactcagcgatctcgtcgttcatcagttatcctgacttgggtaggtccgagcccaaccgaagcaggtcctatattaggttgaagcaactacgcttcaaagggacggtgaagattgaacaggtaccattggcgatgaacatggacggatctacccccaaagtggaaggagtgttctcccttgtcattgttgtggatcgtaaaccacaccttggcccgtctggttgtctgcatacatttgacgagctgtttggtgctaggatacacagtcatggtaacctcagcgtaacccccgcgttgaaagatcgatattacattcgccacgtttgcaaacgtgtgctatctgtggagaaggatacgctgatggtagacgtggaaggatccattcctctctctaaccggcgtattaattgttgggccacgtttaaggacgtggatcgtgagtcatgtaagggtgtttatgataacataagtaagaacgccctgttagtttattactgctggatgtcggatacgcctgcgaaggcatccacttttgtatcgtttgaccttgactatattggttaagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgacttatttcaatgatttggcttgagaagcctgacagttactattgacacattcttggaccgctgtcctgactaattcgttcaactggcccattgacattgtgatgttgggctccgctctctggtcacccacaatcgaagcagattctccggggtctagaaggctggtccccagcctgtttaggtgtctgtatggatggagctcgttctccatttctgagtccgtatctggctgggctgtccctatggtgctcctggaagcccatgattcaccaggcctaatctcaattgggcctcgtagtccaatcctggacatggacgcgcatctgatgggcttcctctcccatttcccataatccacatgggaaaagtccacgtctttttcggagaactgtttggacagtatccttactgttggtgcccggaacgggatgtcgactgagtgtttcgccgtggacaatttcagtttccctttgaacttggcgaagtgggtccgctgatgcacgtttgtatcgcagactttgtagtacaatttccatggaatggggtctttcagcgagaagaacgaagccgagaaatagtggagatctatgttgcatctgatcggaaaagtccacgacgcctgtaaagactcgttgtccgtcattctcttgtcgtggatctccacaatcaccgacccggtggcgttgatcggtacttgttgtctgtattctatgacgcagtgatcgatcttcatgcagctacgactgagtctagctgtcaactgagccgccgtggacggaaattgcagtattatctcagttaggtcatgggaaagttgatattcgtcacggtgtgactcgatgtagttgaaggcgttcggaggatttactaactgagaatccattgtactcgagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 43 - DNA, 2477 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgctcgactagaatgtatggtttgcggaataaacgtggttcatcgttcagccaacgccgattttattcacgtagcagttttttaaatcgcttgtccgctaataagcgtcatgatggcaaacgtcgagctatgaatcctagtaagcccattgacgagcccaagatgtcagcccaacgtatacatgagaaccagtatgggcctgaatttgtaatggcccataattcagccatttctacgtttatcagctaccccagcaagggcaagatggaacccaaccgatcgaggtcctatattaagttgaaacgacttcgtttcaaagggactgtcaagattgaacgtgttcaaccagatatgaacatggacggttctgccccaaaagtggaaggagtgttctctctggtggttgttgtggatcgtaaaccccacttgggtgcgtctggatgcctgcatacattcgacgagctgttcggtgcaaggatccatagccatggtaatctcagcataacaccttctttgaaagaccgattctacataagacacgtgttcaaacgtgtattgtccgtggagaaggatacgatgatggttgacgtggaaggatccacatcgctctctaacaggcgatataattgttggtccacttttaaggatcttgaccatgagtcatgcaagggtgtttatgacaacatcagcaagaacgccctcctagtatattactgttggatgtcagatactatgtcaaaggcatctacttttgtatcgtttgaccttgattatatcggttgagagctcgcccggaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgacttatttcaatgatttggcttgagaagcctgacagttactattgacacattcttggaccgctgtcctgactaattcgttcaactggcccattgacattgtgatgttgggctccgctctctggtcacccacaatcgaagcagattctccggggtctagaaggctggtccccagcctgtttaggtgtctgtatggatggagctcgttctccatttctgagtccgtatctggctgggctgtccctatggtgctcctggaagcccatgattcaccaggcctaatctcaattgggcctcgtagtccaatcctggacatggacgcgcatctgatgggcttcctctcccatttcccataatccacatgggaaaagtccacgtctttttcggagaactgtttggacagtatccttactgttggtgcccggaacgggatgtcgactgagtgtttcgccgtggacaatttcagtttccctttgaacttggcgaagtgggtccgctgatgcacgtttgtatcgcagactttgtagtacaatttccatggaatggggtctttcagcgagaagaacgaagccgagaaatagtggagatctatgttgcatctgatcggaaaagtccacgacgcctgtaaagactcgttgtccgtcattctcttgtcgtggatctccacaatcaccgacccggtggcgttgatcggtacttgttgtctgtattctatgacgcagtgatcgatcttcatgcagctacgactgagtctagctgtcaactgagccgccgtggacggaaattgcagtattatctcagttaggtcatgggaaagttgatattcgtcacggtgtgactcgatgtagttgaaggcgttcggaggatttactaactgagaatccattgtactcgagttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 44 - DNA, 3364 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgagaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

SEQ ID NO: 45 - DNA, 4209 bp, Artificial Sequence
  Synthetic sequence
gttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttg

(19) United States
(12) Patent Application Publication
(10) Pub. No.: US 2021/0115456 A1
(43) Pub. Date: Apr. 22, 2021

(54) EXPRESSION SYSTEMS FOR CELL-TO-CELL GENE TRANSFER IN PLANTS AND METHODS OF USE THEREOF

(71) Applicant: ARIZONA BOARD OF REGENTS ON BEHALF OF ARIZONA STATE UNIVERSITY, Scottsdale, AZ (US)

(72) Inventors: Hugh Mason, Phoenix, AZ (US); Andrew Diamos, Tempe, AZ (US)

(21) Appl. No.: 17/073,102

(22) Filed: Oct. 16, 2020

Related U.S. Application Data
(60) Provisional application No. 62/916,211, filed on Oct. 16, 2019.

(51) Int. Cl. C12N 15/82

(52) U.S. Cl. CPC C12N 15/8203; C12N 15/8258; C12N 15/8251

(57) ABSTRACT

The disclosure relates to compositions comprising a bean yellow dwarf virus (BeYDV) vector that enables cell-to-cell movement of a transgene on a mastrevirus vector using a movement protein and nuclear shutting protein from begomovrius. Such compositions enable transformation of plants using reduced amounts of agrobacterium.

### CROSS REFERENCE TO RELATED APPLICATIONS

This application claims the benefit of and priority to U.S. provisional patent application 62/916,211, filed Oct. 16, 2019, the entirety of the disclosure of which is hereby incorporated by this reference thereto.

### TECHNICAL FIELD

The disclosure relates to geminiviral expression systems modified to enhance transformation efficiency of plant-based expression systems using components from mastrevirus and begomovirus.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 75,015 byte ASCII (text) file named “SeqList” created on Aug. 12, 2020.

### BACKGROUND

Geminiviruses are vector-borne plant viruses with circular ssDNA genomes ranging from 2.5-3.0 kb which cause severe diseases in many economically important crops in tropical and sub-tropical areas (Scholthof et al., 2011). As geminiviruses replicate in plant nuclei, they must cross two barriers to transit between cells: the pores of the nuclear envelope to exit to the cytoplasm, and the intercellular channels, known as plasmodesmata, to spread cell-to-cell (Jeske, 2009; Krichevsky et al., 2006; Lucas, 2006; Waigmann et al., 2004). Though these general barriers must be overcome by all geminiviruses, the specific mechanisms of doing so differ between genera.

The begomoviruses are the largest, most well-studied geminivirus genus, and are divided into the monopartite begomoviruses, which contain only one single-stranded circular DNA component about 2.8 kb in size, and the bipartite begomoviruses, which contain both a DNA-A and DNA-B component. Both DNA-A and DNA-B are small, having genomes strictly between 2.5 and 2.8 kb, which is thought to be required for cell-to-cell movement due to size restrictions imposed by the plasmodesmata (Gilbertson et al., 2003). The DNA-A component produces the replication protein Rep, the coat protein (CP), and auxiliary proteins that regulate transcription, suppress gene silencing, and enhance replication, while the DNA-B component is responsible for virus movement by encoding a nuclear shuttle protein (NSP) and a movement protein (MP) (Hanley-Bowdoin et al., 2013).

Mastreviruses are less well-studied than the begomoviruses, though they result in significant crop damage, especially in sub-Saharan Africa (Shepherd et al., 2007). The mastreviruses have only one small circular single-stranded DNA genome (2.6-2.8 kb) comprising four genes: two replication association proteins, a CP, and a MP. The CP is thought to replace the function of the NSP by shuttling viral replicons out of the nucleus (Liu et al., 1999a). Additionally, the CP is required for ssDNA production for genome packaging (Azzam et al., 1994). After nuclear export, the MP localizes the NSP-DNA complex to the cell periphery for cell-to-cell transport (Jeffrey et al., 1996). The MP from mastreviruses does not seem to bind to DNA directly, instead interacting with the CP-DNA complex. Thus, mastreviruses require the CP for all forms of movement (Liu et al., 1999a).

Exchange of genetic components can alter the host range or tissue specificity of a virus. The susceptibility of plant host species differs drastically between even closely related plant viruses, likely due to the ongoing arms race between plant defenses and virus counter-defenses (Rojas et al., 2005). Cassava mosaic geminivirus, formerly a relatively benign pathogen, underwent recombination to produce a much more severe strain that greatly impeded cassava production in Africa (Legg and Fauquet, 2004). In Spain, a tomato-infecting geminivirus isolated in a wild reservoir was shown to be a recent genetic recombinant between Tomato yellow leaf curl Sardinia virus and Tomato yellow leaf curl virus and had a broader host range than either of the two parent viruses (Garcia-Andres et al., 2006). As a powerful illustration of the potential of geminivirus recombination, it was shown that exchanging the coat protein from African cassava mosaic begomovirus, which is transmitted by whitefly, with the CP of beet curly top curtovirus (BCTV), which is transmitted by leafhopper, allowed the generation of a chimeric virus that had acquired the ability to be transmitted by the BCTV leafhopper vector (Briddon et al., 1990).

Substantial evidence for intra- and inter-species recombination between mastreviruses exist (Kraberger et al., 2013). Nevertheless, in contrast to the many examples of successful complementation experiments reported among members of the begomoviruses that extend cross-genus to the curtoviruses and topocuviruses, there are almost no examples of successful complementation reported for the mastreviruses. Multiple chimeras created between BeYDV and maize streak virus were unable to replicate or infect either host (Liu et al., 1999b). Previous attempts to construct recombinant mastrevirus replicons capable of cell-to-cell or systemic movement with the native CP and MP have not been successful (Palmer and Rybicki, 2001). The only report of viable genetic complementation with a mastrevirus was carried out by swapping the coat and movement protein from similar strains of maize streak virus, which shared high levels of sequence homology (van der Walt et al., 2008).

### SUMMARY

The disclosure relates to plant expression systems that comprise bean yellow dwarf virus-derived (BeYDV-derived) vectors encoding a transgene, wherein the expression system also comprises at least one vector encoding a begomovirus movement protein (MP) and a begomovirus nuclear shuttle protein (NSP) to enable the cell-to-cell movement of the transgene in the transgenic plant. In some embodiments, the plant expression system consists of a chimeric dual replicon vector based on BeYDV that expresses the transgene, the begomovirus MP, and the begomovirus NSP. The expression level of transgene, MP, and NSP are driven by different promoters. In other embodiments, the plant expression system comprises two vectors: one for expressing the transgene and one for expressing begomovirus MP and NSP. In still other embodiments, the plant expression system comprises three expression vectors: one for expressing the transgene, one for expressing the begomovirus MP, and one for expressing the begomovirus NSP.

In one aspect, a T-DNA binary vector for expressing a recombinant protein in a plant cell is disclosed herein. The T-DNA binary vector comprises two replicons, a first long intergenic region (LIR) from bean yellow dwarf virus (BeYDV), a second LIR from BeYDV, and a third LIR from BeYDV. The first replicon is between the first and second LIR from BeYDV and the second replicon is an expression cassette and is between the second and third LIR from BeYDV. The first replicon comprises a first sequence encoding a begomovirus MP, a second sequence encoding a begomovirus NSP, and a first short intergenic region (SIR) from BeYDV. The first SIR separates the first and second sequences. The second replicon comprises a third sequence encoding a transgene. In some aspects, the size of each of the replicons is between 2 kb and 3.1 kb.

In some embodiments, the begomovirus MP and the begomovirus NSP are both from BeYDV. In other embodiments, the begomovirus MP and the begomovirus NSP are both from abutilon mosaic virus (AbMV). In yet other embodiments, the begomovirus MP is from AbMV and the begomovirus NSP is from BeYDV. In still other embodiments, the begomovirus MP is from BeYDV and the begomovirus NSP is from AbMV.

In some embodiments, the expression vector further comprises a second SIR from BeYDV and a fourth sequence encoding Rep/RepA from BeYDV. The fourth sequence is downstream of the third sequence. The second SIR separates the third and fourth sequences.

In some embodiments, the second replicon further comprises 5′ untranslated region (UTR) from tobacco mosaic virus (TMV 5′), and the TMV 5′ is upstream of the third sequence.

In some embodiments the second replicon further comprises an intronless form of the gene terminator from tobacco extension (Ext 3′), and the Ext 3′ is downstream of the third sequence. In some aspects, the Ext 3′ is also upstream of the second SIR.

In some embodiments, the TATA box of at least one of the first, second, and third LIR is mutated and comprise the nucleic acid sequence TATAAG.

In some embodiments the first replicon further comprises a truncated pinII terminator downstream of the second sequence encoding the begomovirus NSP, wherein the first LIR from BeYDV is a v-sense LIR comprising a mutated TATA box having the sequence TATAAC and is upstream of the second sequence encoding the begomovirus NSP, and the second LIR from BeYDV is a c-sense LIR and is upstream of the first sequence encoding the begomovirus MP.

In another aspect, the disclosure relates to a plant expression system comprising three expression vectors for facilitating cell-to-cell movement of a transgene. The first expression vector has a T-DNA region a replicon spanning a first LIR from BeYDV and a second LIR from BeYDV. The first replicon comprises a first sequence encoding a transgene, a 5′ UTR upstream of the first sequence, a terminator downstream of the first sequence, a second sequence encoding Rep/RepA from BeYDV, and a SIR from BeYDV. The first sequence, the 5′ UTR, and the terminator are upstream of the SIR, and the second sequence is downstream of the SIR. The second expression vector has a T-DNA region comprising a promoter selected from cauliflower mosaic virus 35S promoter or agrobacterium NOS promoter, a third sequence encoding a begomovirus MP; and a terminator selected from agrobacterium NOS termination or a truncated form of the gene terminator from tobacco extension (Ext 3′). The third expression vector has a T-DNA region comprising a promoter selected from cauliflower mosaic virus 35S promoter or agrobacterium NOS promoter; a fourth sequence encoding a begomovirus NSP; and a terminator selected from agrobacterium NOS terminator or Ext 3′. In some aspects, the size of each of the replicons is between 2 kb and 3.1 kb.

In some embodiments the 5′ UTR in the first expression vector comprises 5′ UTR from tobacco mosaic virus.

In some embodiments, the terminator in the first expression system comprises Ext 3′.

In some embodiments, the begomovirus MP and the begomovirus NSP are both from BeYDV. In other embodiments, the begomovirus MP and the begomovirus NSP are both from AbMV. In yet other embodiments, the begomovirus MP is from AbMV and the begomovirus NSP is from BeYDV.

In some embodiments, the TATA box of at least one of the third LIR and the fourth LIR is mutated and comprises the nucleic acid sequence TATAAG.

In yet another aspect, the disclosure relates to a plant expression system comprising a dual replicon vector for facilitating cell-to-cell movement of a transgene comprising a first expression vector with a T-DNA region comprising a first replicon spanning a first LIR from BeYDV and a second LIR from BeYDV and a second expression vector with a T-DNA region comprising a second replicon spanning a third LIR from BeYDV and a fourth LIR from BeYDV. The first replicon comprises a first sequence encoding a transgene, a 5′ UTR upstream of the first sequence, a terminator downstream of the first sequence, a second sequence encoding Rep/RepA from BeYDV, and a first SIR from BeYDV. The first sequence, the 5′ UTR, and the terminator are upstream of the short intergenic region and the second sequence is downstream of the first SIR. The second replicon comprises a third sequence encoding a begomovirus MP, a fourth sequence encoding a begomovirus NSP, and a second SIR from BeYDV. The second SIR separates the second and third sequence. In some aspects, the size of each of the replicons is between 2 kb and 3.1 kb.

In some embodiments, the begomovirus MP and the begomovirus NSP are both from BeYDV. In other embodiments, the begomovirus MP and the begomovirus NSP are both from AbMV. In yet other embodiments, the begomovirus MP is from AbMV and the begomovirus NSP is from BeYDV.

In some embodiments the TATA box of the third LIR and/or fourth LIR is mutated and comprises the nucleic acid sequence TATAAG.

In another aspect, the disclosure relates to a plant expression system comprising a dual replicon vector for facilitating cell-to-cell movement of a transgene using begomovirus MP and begomovirus NSP. The dual replicon comprises a first expression vector and a second expression vector, where the expression vectors separately express either begomovirus MP or begomovirus NSP. The first expression vector has a T-DNA region comprising a first replicon that spans a first LIR from BeYDV and a second LIR from BeYDV. The first replicon comprises a first sequence encoding a transgene, a second sequence encoding either the begomovirus MP or the begomovirus NSP, and a SIR from BeYDV that separates the first sequence and the second sequence. The second expression vector has a T-DNA region comprising a second replicon that spans a third LIR from BeYDV and a fourth LIR from BeYDV. The second replicon comprises a third sequence encoding either the begomovirus MP or the begomovirus NSP.

In some embodiments, the begomovirus MP and the begomovirus NSP are both from BeYDV. In other embodiments, the begomovirus MP and the begomovirus NSP are both from AbMV. In yet other embodiments, the begomovirus MP is from AbMV and the begomovirus NSP is from BeYDV.

The disclosure also relates to improved methods of producing transgenic plants. The method comprises transforming the plants with an expression system or binary vector described herein using lower amount of agrobacterium, for example, at a concentration as measured by OD₆₀₀ of less than 0.2, about 0.02, or about 0.002.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C depict, according to certain embodiments, the construction of a mastreviral reporter system. FIG. 1A depicts a generalized schematic representation of the T-DNA region of recombinant mastreviral genomes expressing GFP. RB and LB refers to the right and left borders of the agrobacterium T-DNA region; LIR refers to the long intergenic region from BeYDV; V1 refers to the movement protein gene from BeYDV; V1mut refers to the V1 with mutated start codon; IS refers to the small intergenic sequence between the BeYDV movement and coat protein genes; GF refers to the plant-optimized coding sequence of green fluorescent protein; Ext 3′ refers to a truncated form of the gene terminator from tobacco extensin; SIR refers to the short intergenic region from BeYDV; Rep/RepA refers to the replication associated genes from BeYDV; and TMV5′ refers to the 5′ UTR from tobacco mosaic virus. FIG. 1B shows exemplary leaves of Nicotiana benthamiana that were agroinfiltrated with the indicated vectors and representative images showing GFP fluorescence at 4 DPI. FIG. 1C is an image on an ethidium bromide-stained agarose gel of total DNA (1 μg) extracted from N. benthamiana leaves agroinfiltrated with pBYR-L5GFP. The annotations mark bands corresponding to viral replicons and plant chromosomal DNA.

FIGS. 2A-2F depict, in accordance with certain embodiments, the coexpression of BDMV MP and NSP in N. benthamiana leaves using mastreviral replicons. FIG. 2A shows a western blot detecting 35S-MP or 35 S-NSP in leaves of N. benthamiana agroinfiltrated with HA-tagged 35S-MP or 35S-NSP using HA-specific antibodies. Mock samples were infiltrated with the empty vector pPS1. FIGS. 2B-2F depicts photographs of leaf spots coinfiltrated with the mastreviral replicon vector pBYR-L5GFP and either pPS1 or the indicated vectors. Representative leaves were photographed after 3-5 days post-infiltration (DPI) under UV light to monitor GFP fluorescence or under visible light to monitor leaf health.

FIGS. 3A-3D depict, according to certain embodiments, the characterization of GFP expression by chimeric BDMV/BeYDV dual replicon vectors. FIG. 3A depicts a exemplary schematic of the general arrangement of chimeric BDMV/BeYDV dual replicon vectors (pBYBDx) that allows replicational release of a MP/NSP-containing replicon and a separate GFP/Rep replicon identical to pBYR-L5GFP. The components F1/F2 and T1/T2 refer to flanking region modifications and terminator insertions respectively, as described in Table 1. FIG. 3B depicts photographs of leaves agroinfiltrated at the OD₆₀₀ values of 0.2 or 0.02 as indicated with either chimeric vectors or pBYR-L5GFP as control were imaged at 4 DPI (left) or 6 DPI (right). FIGS. 3C and 3D depicts quantifications of GFP expression from protein extracts of agroinfiltrated leaves. Protein extracts were separated by mass using SDS-PAGE and GFP expression was determined by quantification of the fluorescent band using ImageJ software. Columns represent means±standard deviation of 3 independently infiltrated samples. Three stars (***) indicates p<0.001 by student's t-test.

FIGS. 4A-4D depict, in accordance with certain embodiments, results from fluorescence microscopy of N. benthamiana leaves agroinfiltrated with chimeric BeYDV/BDMV replicons. The leaves were agroinfiltrated with chimeric BeYDV/BDMV replicon vectors (pBYBD—shown in Table 1) or T-DNA vectors supplying BDMV DNA-A and DNA-B Representative images were taken at 6 DPI showing individual fluorescent cells seen predominately without MP/NSP expression (FIG. 4A) or clusters of fluorescent cells seen predominately with MP/NSP expression (FIG. 4B). FIG. 4C is a graph showing the average sizes of fluorescent cell clusters, while FIG. 4D is a graph showing the proportion of individual fluorescent cells to clustered fluorescent cells were counted. Columns represent means±standard deviation of more than 50 cell counts from two independent experiments.

FIGS. 5A and 5B depict, in accordance with certain embodiments, the limiting effect of replicon size in vectors with BDMV MP and NSP. FIG. 5A shows images taken under UV light at 4 DPI (left) and 8 DPI (right) of leaves that were agroinfiltrated with either pBYBDf, containing the 2.5 kb GFP replicon from pBYR-L5GFP, or pBYBDf3.9, containing a 3.9 kb GFP replicon with two insertions: (1) the 35S promoter with duplicated enhance regions driving GFP, and (2) the intronless tobacco extensin terminator. Both vectors contain identical 2.7 kb MP/NSP replicons. FIG. 5B shows leaves agroinfiltrated with either pBYBDf3.0 (3 kb GFP replicon), which contains an inserted 35S promoter driving GFP, or pBYBDf (2.5 kb GFP replicon) and imaged under UV light at 8 DPI.

FIGS. 6A-6D depict, in accordance with certain embodiments, the characterization of AbMV MP and NSP with BeYDV replicons. The strong 35S promoter was used to produce AbMV NSP (pABV1e) and MP (pABC1e) in plant leaves. Coinfiltration of AbMV MP and NSP with pBYR-L5GFP is shown under UV light (FIG. 6A) or in visible light (FIG. 6B) after 4 DPI. FIG. 6C depicts a representative leaf imaged under UV light at 6 DPI agroinfiltrated with hybrid vectors that contain the same genetic elements as pBYBDb (see Table 1) with the following modifications: pBYAMa contains the AbMV MP and NSP directly replacing the BDMV MP and NSP, while pBYAMb contains the AbMV MP replacing BDMV MP, but retains the BDMV NSP. These vectors were agroinfiltrated at a low agrobacterium OD₆₀₀ of 0.002. FIG. 6D shows the average sizes of fluorescent cell clusters. Columns represent means±standard deviation of more than 50 cell counts from two independent experiments.

FIGS. 7A and 7B depict, in accordance with certain embodiments, NSP inhibits replicon accumulation and induces necrosis. Leaves of N. benthamiana were agroinfiltrated with 35S-NSP (pBV1e) and 35S-MP (pBC1e). Total DNA was extracted and analyzed by agarose gel electrophoresis for replicon formation (FIG. 7A). FIG. 7B shows tissue cell death in representative leaves at 8 DPI.

FIGS. 8A and 8B depict, in accordance with certain embodiments, that mutation of LIR TATA box reduces gene expression but not replicon accumulation. Leaves were agroinfiltrated with either pBYR-L5GFP containing the wildtype LIR TATA-box or with a vector containing a single nucleotide change (TATAAA to TATAAG) and characterized for GFP expression by agroinfiltrated leaf spots at 4 DPI (FIG. 8A) and replicon accumulation at 4 DPI (FIG. 8B).

FIG. 9 depicts the plasmid map for pBYR-L5GFP.

FIG. 10 depicts the plasmid map for pBYBDa.

FIG. 11 depicts the plasmid map for pBYBDb.

FIG. 12 depicts the plasmid map for pBYBDc.

FIG. 13 depicts the plasmid map for pBYBDd.

FIG. 14 depicts the plasmid map for pBYBDf.

FIG. 15 depicts the plasmid map for pBYBDh.

FIG. 16 depicts the plasmid map for pBYBDi.

FIG. 17 depicts the plasmid map for pBYBDk.

FIG. 18 depicts the plasmid map for pBYBDl.

FIG. 19 depicts the plasmid map for pBYBDm or pBYBDM.

FIG. 20 depicts the plasmid map for pBYBDp.

FIG. 21 depicts the plasmid map for pBYBDq.

FIG. 22 depicts the plasmid map for pBYBDr.

FIG. 23 depicts the plasmid map for pBYAMa.

FIG. 24 depicts the plasmid map for pBYAMb.

FIG. 25 depicts the plasmid map for pBYBDf3.0.

FIG. 26 depicts the plasmid map for pBYBDf3.9.

FIGS. 27A-27E depict, in accordance with certain embodiments, the different vector designs and vector compositions of the described plant expression system. As used in the figures, RB and LB refers to the right and left borders of the agrobacterium T-DNA region; LIR refers to the long intergenic region from BeYDV; SIR refers to the short intergenic region from BeYDV; GFP refers to the plant-optimized coding sequence of green fluorescent protein, which can be substituted with any other gene of interest; Ext 3′ refers to a truncated form of the gene terminator from tobacco extensin; Rep/RepA refers to the replication associated genes from BeYDV; and TMV5′ refers to the 5′ UTR from tobacco mosaic virus. FIG. 27A depicts an exemplary chimeric dual replicon vector. FIG. 27B depicts an exemplary system with three separate vectors: one for encoding GFP or gene of interest, one for encoding NSP, and one for encoding MP. FIG. 27C depicts an exemplary system with two separate vectors: one for encoding GFP or gene of interest and one for expressing NSP and MP. FIG. 27D depicts an exemplary system with two separate vectors: one for encoding GFP or the gene of interest and NSP and one for encoding GFP or the gene of interest and MP. FIG. 27E depict an exemplary system with two separate vectors: one for encoding GFP or the gene of interest and either NSP or MP and one for encoding the other of NSP or MP.

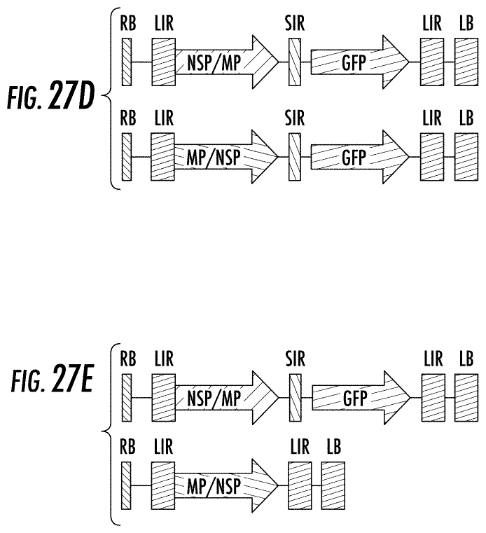

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to a deviation no more than 10% of the given value, for example, a deviation of 10%, 5%, 1%, or 0.5% of the given value.

As used herein the term” expression vector” refers to a plasmid used to introduce a specific gene, for example a transgene, into a target cell and use the target cell's own mechanism of protein expression to produce the protein encoded by the specific gene. As used herein, the terms “bean yellow dwarf virus vector”, “BeYDV vector,” “BeYDV-based vector,” or a vector of the “BeYDV system” refer to an expression vector that comprises the nucleic acid sequence of BeYDV's long intergenic regions (LIRs) and short intergenic region (SIR). A replication-competent BeYDV vector further comprises the nucleic acid sequence for Rep/RepA.

As used herein, the term “expression cassette” refers to a distinct component of vector DNA, which contains gene sequences and regulatory sequences to be expressed by the transfected cell. An expression cassette comprises three components: a promoter sequence (part of the 5′ untranslated region, 5′ UTR), an open reading frame, and a 3′ untranslated region (3′ UTR). In some aspects, the regulatory sequences are found in the 5′ UTR and the 3′ UTR.

As used herein, the term “replicon cassette” refers to an expression cassette comprising at least one gene that assists with replication of an organism. For example, in certain embodiments, the expression vector disclosed herein comprise a replicon cassette comprising a sequence encoding Rep, RepA, or Rep/RepA from BeYDV. A vector that comprises a sequence encoding Rep, RepA, or Rep/RepA from BeYDV is also referred to herein as a vector that is “replication-competent.”

As used herein, the term “replicon” refers to a portion of a vector between two intergenic regions. As used herein, a “mastrevirus replicon” refers to a portion of the vector where two long intergenic region (LIR) from BeYDV designate the borders of the replicon.

As used herein, the term “transgene” refers to a gene from one organism that is introduced into another organism.

The disclosure relates to expression systems that enable cell-to-cell movement of a transgene in plants, in particular transgene in a mastrevirus replicon. The expression systems utilize the movement protein (MP) and nuclear shuttle protein (NSP) from bipartite begomovirus with the transgene being expressed on a replicon based on the mastrevirus bean yellow dwarf virus (BeYDV). In particular embodiments, the expression systems comprise a BeYDV replicon that supplies begomovirus MP and NSP from the native LIR promoter to provide the protein machinery for cell-to-cell movement of a transgene that is supplied in a replication-competent BeYDV vector.

Both the MP and NSP from begomoviruses have been shown to bind DNA in a sequence non-specific manner that instead depends on form and size. Though some differences have been reported between species, in general, genome-sized DNA molecules are efficiently transported (Hehnle et al., 2004; Rojas et al., 1998). Interestingly, the NSP is not required for virus infectivity with several begomoviruses, likely due to overlapping function with the CP (Sudarshana et al., 1998; Zhou et al., 2007). The DNA-A component of the bipartite begomoviruses can spread systemically in the absence of DNA-B, though in a very limited capacity (Hou et al., 1998). Inside the nucleus, viral genomes have been shown to interact with NSP and histone H3 (Zhou et al., 2011). The current model of intracellular movement is that a complex of histone H3, NSP, and viral genomic DNA is exported out of the nucleus, where it interacts with the MP. The MP-NSP-genome-histone H3 complex is then trafficked to the cell periphery and through the plasmodesmata to neighboring cells (Krenz et al., 2010). Though the exact mechanism of transport is not fully understood, heat shock cognate 70 kDa protein has been implicated in the movement of viral replicons through a network of stromules which are formed upon infection and connect the nucleus, plastids, and plasmodesmata (Krenz et al., 2012). The NSP is also responsible for nuclear import of replicated viral genomes in neighboring cells (Krichevsky et al., 2006)

As described herein, expression of bean dwarf mosaic virus (BDMV) MP and NSP separately at controlled levels allowed efficient cell-to-cell movement of recombinant mastreviral replicons in Nicotiana benthamiana leaves. The MP and NSP from the phloem-limited geminivirus Abutilon mosaic virus (AbMV) could efficiently move BeYDV replicons when expressed at appropriate levels. Accordingly, the MP and NSP are both from BDMV in some embodiments of the described expression system. In other embodiments, the MP and NSP are from AbMV. In still other embodiments, the MP and NSP is from different bipartite begomoviruses, for example one of the two proteins is from BDMV while the other is from AbMV. In certain embodiments, the MP is from AbMV and the NSP is from BDMV.

Efficient cell-to-cell movement in planta strongly depends on the relative expression levels of MP and NSP. Overexpression of MP and NSP causes leaf cell death, but production of functionally relevant levels of MP and NSP does not cause notable symptoms with BeYDV replicons. Accordingly, the efficiency of cell-to-cell movement is greatly improved by optimizing the vector size and expression level of MP and NSP. MP and NSP expression needs to be fine-tuned to strike a balance between providing efficient cell-to-cell movement capabilities to new viral replicons, while still allowing sufficient replicon availability in the nucleus for viral transcription, as well as avoiding triggering the plant hypersensitive response.

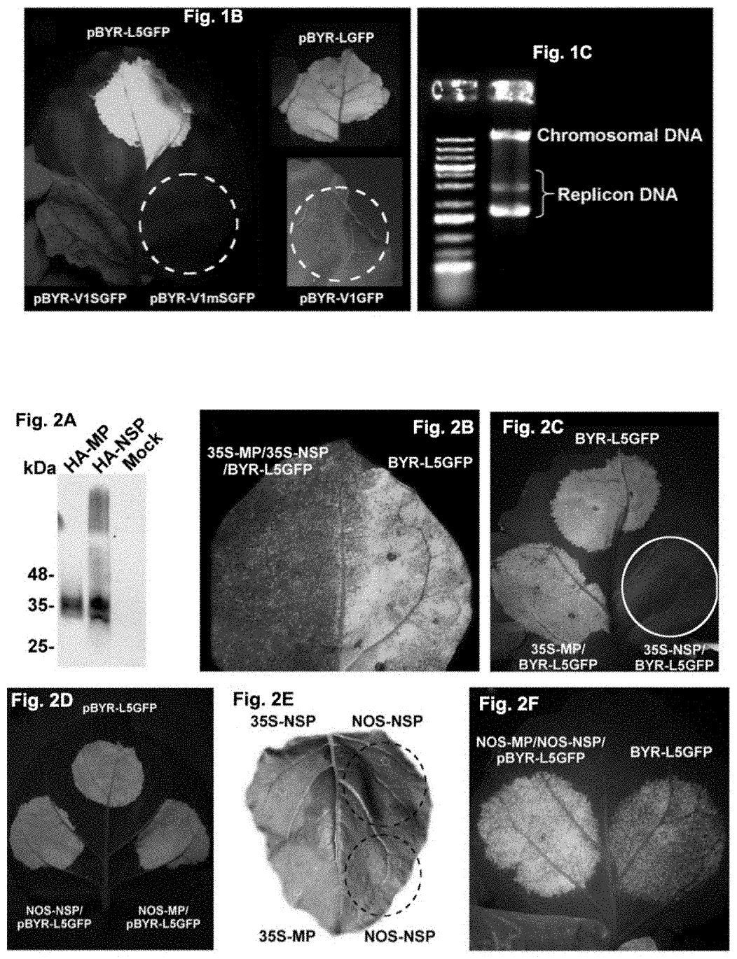

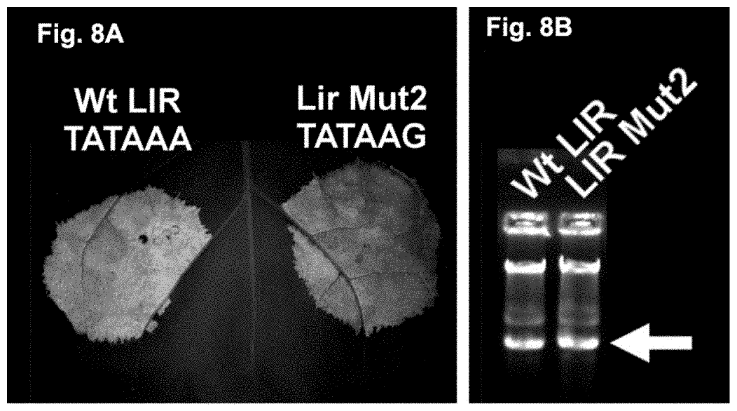

BDMV NSP strongly inhibited GFP replication and expression from genome-sized BeYDV replicons when produced from the strong 35S promoter (FIGS. 2B, 2C, and 7A), suggesting that NSP efficiently interacts with mastreviral replicons. High levels of NSP may reduce the availability of transcription templates, either by transport out of the nucleus or by interfering with replication, as NSP binds most DNA forms at high concentrations, forming collapsed structures (Hehnle et al., 2004; Rojas et al., 1998). Increased cell death was also observed with overexpression of NSP (FIGS. 2E and 7B); however, GFP inhibition occurred many days prior to the development of leaf necrosis. Expression of NSP from a weaker promoter did not appreciably impact GFP expression (FIG. 2D) and in support of the general sequence-nonspecific transport capabilities of the BDMV MP and NSP to move genome-sized DNA molecules, coexpression of each by the NOS promoter caused an increase in GFP fluorescent area produced by recombinant mastreviral replicons (FIG. 2F). Accordingly, in certain embodiments, the promoter driving MP or NSP expression is selected from the group consisting of: strong cauliflower mosaic virus 35S promoter, v-sense long intergenic region from BeYDV, c-sense long intergenic region from BeYDV, and agrobacterium nopaline synthase (NOS) promoter. In some aspects, the LIR is mutated at the TATA box (for example, the mutated TATA box sequence is TATAAG) or at the start codon context sequence (for example, the mutated start codon context sequence is GGAATG). In some embodiments, the expression of MP and/or NSP is further modified at the 5′ region with the tobacco mosaic virus 5′ UTR (TMV 5′) or the tobacco etch virus 5′ UTR (TEV 5′). The expression of MP and/or NSP may also be modified at the 3′ region, for example, with truncated 3′ UTR of tobacco extension gene (also known as intronless 3′ UTR of tobacco extension gene, Ext 3′). The expression of MP and/or NSP may additionally be modified with at least one terminator selected from the group consisting of: short intergenic region (SIR) from BeYDV, the potato proteinase inhibitor II terminator (pinII), the soybean vegetative storage protein B terminator (VspB), the tobacco intronless extensin terminator (ext), and the 35S terminator from cauliflower mosaic virus (35S).

The ability to confer cell-to-cell movement to mastrevirus replicons in the expression systems described herein depend strongly on replicon size. MP was also shown to facilitate cell-to-cell movement of appropriately sized plasmid DNA (Gilbertson et al., 2003). As shown in the Examples, replicons between the sizes of 2.1 kb and 2.7 kb replicons were efficiently moved, while replicons larger that 3.1 kb were inhibited. Replicons with a size of 3.9 kb were very strongly inhibited.

Because these expression systems enable cell-to-cell movement of a transgene, a plant can be more efficiently transformed even with lower amounts of agrobacterium. Accordingly, the disclosure also relates to improved methods of producing a transgenic plant by administering agrobacterium in a concentration of OD₆₀₀ less than 0.2 to the plant. In certain embodiments, the concentration of agrobacterium is OD₆₀₀ of about 0.02, 0.002, or 0.0002. In particular embodiments, the plant is administered agrobacterium transformed with the chimeric dual replicon vector or chimeric expression system described herein.

### 1. Chimeric Dual Replicon Vector

In some implementations, the expression system consists of a chimeric dual replicon vector. The chimeric vector is a BeYDV vector comprising three long intergenic regions from BeYDV (LIRs) which form two replicons, a first replicon and a second replicon. The first replicon spans the region between the first LIR and the second LIR, while the second replicon spans the region between the second LIR and the third LIR.

The first replicon comprises a first sequence encoding a begomovirus MP and a second sequence encoding a begomovirus NSP. A short intergenic region from BeYDV (SIR) separates the first sequence and the second sequences. Accordingly, the first replicon comprises a first LIR, the first sequence encoding a begomovirus MP, the SIR, and the second sequence encoding a begomovirus NSP. In some aspects, the first LIR is a v-sense LIR while the second LIR is a c-sense LIR, and the arrangement of the first replicon has the first LIR driving the expression of the begomovirus NSP and the second LIR driving the expression of the begomovirus MP. In such embodiments, the expression of the begomovirus may be further modified by TMV 5′. In such embodiments, the first LIR may be mutated at the TATA box or the start codon context box as described above. In other aspects, the arrangement of the first replicon has the first LIR driving the expression of the begomovirus MP and the second LIR driving the expression of the begomovirus NSP. In some embodiments, the first replicon is replication-competent and thus comprises a sequence encoding Rep/RepA from BeYDV. Accordingly, the first replicon may further comprise another SIR to separate the sequence encoding Rep/RepA from the sequences encoding the begomovirus MP and NSP.

The second replicon is an expression cassette that comprises a third sequence encoding a transgene. In certain embodiments, the second replicon is replication-competent and further comprises a fourth sequence encoding Rep/RepA from BeYDV, which is separated from the third sequence encoding the transgene with a second SIR. In some aspects, the translation of the third sequence encoding the transgene is modified with the 3′ UTR of tobacco extensin gene, whether intronless (Ext 3′) or the full 3′ UTR. Accordingly, in certain embodiments, the second replicon comprises a third sequence encoding the transgene and Ext 3′, where Ext 3 is downstream of the third sequence encoding the transgene but upstream of the second SIR. The translation of the third sequence encoding the transgene may also be modified with a 5′ UTR, for example, PsaK truncated 5′ UTR (PsaK 5′).

In some embodiments, the arrangement of the chimeric dual replicon vector is LIR-MP-SIR-Rep/RepA-LIR-NSP-SIR-transgene-LIR.

Optimized chimeric vectors containing a BeYDV replicon supplying BDMV or AbMV MP and NSP from the native LIR promoter provided cell-to-cell movement to replication-competent BeYDV GFP vectors (see Table 1), comparable to or exceeding the movement efficiency of wildtype BDMV replicons. These vectors result in rapid accumulation of very high levels of GFP even when typical agrobacterium concentrations are substantially reduced.

### 2. Chimeric Expression System

In other implementations, the expression system comprises a plurality of expression vectors, each comprising at least one replicon. Among the plurality of expression vectors, at least one is a BeYDV vector and comprises a sequence encoding a transgene. In certain embodiments, the BeYDV vector comprising the sequence encoding the transgene is replication-competent, thus comprising a second sequence encoding Rep/RepA of BeYDV that is separated by from the sequence encoding the transgene with a SIR. In some aspects, the BeYDV vector comprising the sequence encoding the transgene further comprises a 5′ UTR, a terminator, or both that modify the translation of the transgene. For example, the 5′ UTR may be TMV 5′ or PsaK 5′, and the terminator may be Ext 3′. The other expression vectors in the plurality of expression vectors express begomovirus MP and NSP. In some aspects, expression system comprises two expression vectors that comprise a sequence encoding a transgene. While one vector further comprises a sequence encoding begomovirus NSP, the other vector further comprises a sequence encoding begomovirus MP. In such embodiments, the sequence encoding the transgene is separated by the sequence encoding the begomovirus NSP or MP by an intergenic region, for example SIR.

LIR is a bidirectional promoter that acts differently at different times during infection: initially, the early part of the promoter makes Rep/RepA first after infection, and then later it makes the native movement/coat proteins (Rep/RepA activate the late promoter, and suppress the early promoter). In some aspects, the expression of the MP and NSP are driven by the late promoter, thus the sequence encoding MP or NSP is downstream of the sequence encoding the transgene. In other aspects, the expression of the transgene is driven by the late side promoter, thus the sequence encoding MP or NSP is upstream of the sequence encoding the transgene.

In some embodiments, the expression system further comprises a second expression vector comprises a sequence encoding begomovirus MP, while a third expression vector comprises a sequence encoding begomovirus NSP. In such embodiments, the expression of the begomovirus MP and NSP may be driven by a strong promoter like 35S or weaker promoter like NOS (see Examples 3 and 7). The expression of begomovirus MP and NSP may be modified with 5′ UTRs and terminators. Thus, the second and third expression vectors each may further comprise a 5′ UTR, a terminator, or both to modify the translation of the MP and NSP, for example, the 5 UTR may be TEV 5′ and the terminator may be vspB. In some implementations, the second and third expression vectors are replication-competent. Accordingly, such embodiments of the second and third expression vector further comprise a sequence encoding Rep/RepA from BeYDV.

Accordingly, in certain embodiments, the expression system comprises a first expression vector with a T-DNA region comprising a replicon spanning a first long intergenic region from BeYDV and a second long intergenic region from BeYDV, wherein the replicon further comprises a first sequence encoding transgene, a 5′ UTR upstream of the first sequence, a terminator downstream of the first sequence, a second sequence encoding Rep/RepA from BeYDV; and a short intergenic region from BeYDV; a second expression vector with a T-DNA region comprising a promoter selected from the group consisting of: cauliflower mosaic virus 35S promoter and agrobacterium NOS promoter, a third sequence encoding a begomovirus movement protein, and a terminator comprising a terminator selected from the group consisting of: agrobacterium NOS terminator and a truncated form of the gene terminator from tobacco extension (Ext 3′); and a third expression vector with a T-DNA region comprising a promoter selected from cauliflower mosaic virus 35S promoter or agrobacterium NOS promoter, a fourth sequence encoding a begomovirus nuclear shuttle protein, a terminator selected from agrobacterium NOS terminator or Ext 3′. For the first expression vector, the first sequence, the 5′ UTR, and the terminator are upstream of the short intergenic region and the second sequence is downstream of the short intergenic region. For all three expression vectors, the size of the first replicon or of the T-DNA regions is preferably between 2 kb and 3.1 kb.

In other embodiments, the expression system further comprises a second expression vector based on BeYDV that expresses both begomovirus MP and begomovirus NSP. In some aspects, the second expression vector is also replication-competent. The expression vector comprises a sequence that encodes begomovirus MP and a sequence that encodes begomovirus NSP that are separated by an intergenic region, for example, SIR. The expression of the begomovirus MP and the begomovirus NSP is driven by a LIR in the second expression vector. Accordingly, the second expression vector comprises two LIRs, and the sequence that encodes begomovirus MP and the sequence that encodes begomovirus NSP are flanked by the LIRs. In some aspects, a c-sense LIR drives the expression of the MP, while a v-sense LIR drives the expression of the NSP.

Thus, in certain embodiments, the expression system comprises a first expression vector and a second expression vector. The first expression vector has a T-DNA region comprising a first replicon spanning a first long intergenic region from bean yellow dwarf virus (BeYDV) and a second long intergenic region from BeYDV. The first replicon further comprises a first sequence encoding transgene, a 5′ UTR upstream of the first sequence, a terminator downstream of the first sequence, and a second sequence encoding Rep/RepA from BeYDV, and a first short intergenic region from BeYDV. The first sequence, the 5′ UTR, and the terminator are upstream of the short intergenic region, and the second sequence is downstream of the first short intergenic region. The second expression vector has a T-DNA region comprising a second replicon spanning a third long intergenic regions from BeYDV and a fourth long intergenic region from BeYDV. The second replicon further comprises a third sequence encoding a begomovirus movement protein, a fourth sequence encoding a begomovirus nuclear shuttle protein, and a second short intergenic region from BeYDV, wherein the second short intergenic region separates the second and the third sequences. In preferred embodiments, the size of the first replicon and the size of the second replicon is between 2 kb and 3.1 kb.

### Illustrative, Non-Limiting Example in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

### 1. Vector Construction

A mastreviral vector with GFP replacing the BeYDV CP was created by overlap extension PCR. Primers LIRAscI (5′-TGGCGCGCCGCTCTAGCAGAAGGCATGTTG, SEQ ID NO. 1) and GFP-V2-R (5′-TCGCCCTTGCTCACCATGATGCACCCCGCCTA, SEQ ID NO. 2) were used to amplify the LIR-MP from pBY002 (Mor et al., 2003) and combined by overlapping GFP segments amplified with primers GFP-V2-F (5′-GTAGGCGGGGTGCATCATGGTGAGCAAGGG CGA, SEQ ID NO. 3) and VSPHT (5′-TGAATAGTGCATATCAGCATACCTTA, SEQ ID NO. 4) from pBY-GFP (Huang et al., 2009). The final vector pBYR-V1SGFP was assembled by three fragment ligation: the vector backbone from pBY-GFP digested with AscI-EcoRI, the overlap PCR fragment digested with AscI-SacI, and the truncated extensin terminator from pBY027.Eu2 (Rosenthal et al., 2018) digested with SacI-EcoRI. A construct pBYR-V1GFP fusing GFP directly downstream from MP without the intergenic sequence upstream from the CP was created similarly, using overlapping primers GFP-V1-R (5′-TCGCCCTTGCTCACCATCTAC GGTCCTGGATGATCC, SEQ ID NO. 5) and its complementary primer. To mutate the start codon of MP, overlapping mutagenic primer V1-Mut-R (5′-CAATATACGCTTTATCAAATACCATCAC, SEQ ID NO. 6) and its complement were used with LIRascI and Ext3-R (5′-CTTCTTCTTCTTC TTTTCTCATTGTC, SEQ ID NO. 7) to amplify an LIR-V1mut-GFP segment from pBYR-V1SGFP. The product was digested with AscI-SacI and ligated into pBYR-V1SGFP digested likewise to yield pBYR-V1mutSGFP. A mastreviral replicon fusing GFP directly downstream from the LIR promoter was created by PCR amplifying the LIR from pBY-GFP with primer LIR-Xho (5′-AACTCGAGCAAATACCATCACATCG, SEQ ID NO. 8), designed to insert a XhoI site upstream from the MP start codon, and primer PNOS-Xho-R (5′-GGCTCGAGTTTGGATTGA GAGTGAATATGAGAC, SEQ ID NO. 9). The final vector pBYR-L5GFP was assembled by three fragment ligation: the vector backbone pBYR-V1GFP digested with AscI-SacI, the PCR product digested with AscI-XhoI, and the XhoI-SacI GFP fragment with TMV 5′ UTR from pBYR2e-GFP (Diamos et al., 2016). The construct pBYR-LGFP, which has the TMV 5′ UTR deleted, was created by XhoI-XbaI digestion of pBYR-L5GFP, blunting the ends with the Klenow fragment of DNA polymerase, and self-ligating the backbone vector.

Vectors pBDA1.5-GFP containing the BDMV DNA-A with coat protein replaced by GFP (Sudarshana et al., 1998). NSP was amplified from pBDB1.5 with primers BV1-Xba-F (5′-gaTCTAGAATGTATGGTTTGCGGAATAAAC, SEQ ID NO. 10) and BV1-Sac-R (5′-ccGAGCTCT CAACCGATATAATCAAGGTCAAAC, SEQ ID NO. 11) designed to insert XbaI and SacI sites flanking the NSP gene. The amplification product was digested with XbaI-SacI and ligated into pGFPe-TMV (Diamos et al., 2016), an agrobacterium T-DNA expression vector containing the strong 35S promoter and the P19 suppressor of RNA silencing, digested likewise to yield pBV1e (referred to as 35S-NSP). The BDMV MP was amplified from pBDB1.5 with primers BC1-Xba-F (5′-ggTCTAGAATGGATTCTCAATTGGTCAATC, SEQ ID NO. 12) and BC1-Sac-R (5′-gcGAG CTCTTATTGCAACGATTTGGGCT, SEQ ID NO. 13), digested with XbaI-SacI, and ligated into pGFPe-TMV digested likewise to create pBC1e (referred to as 35S-MP).

To create chimeric mastrevirus replicons containing the BDMV MP and NSP, a preliminary cloning vector was created by four fragment ligation: from pBYRL5-GFP, the AscI-XhoI fragment (LIR-TMV-GFP) and EcoRI-FseI fragment (SIR-Rep-LIR) were excised and, along with the SacI-EcoRI potato pinII terminator fragment from pHB114 (Richter et al., 2000), ligated into pBY027 (Mor et al., 2003) digested with AscI-FseI. To create a vector with only the BeYDV bidirectional SIR terminator but with available SacI-EcoRI sites for further terminator insertions, the potato pinII terminator was deleted by digestion with SbfI-SmaI, blunting the ends with Klenow fragment of DNA polymerase, and self-ligating the backbone. Then, BDMV NSP was excised from pBV1e by XhoI-SacI digestion and ligated into this new vector digested likewise to yield pBYR0-LBV1. The initial MP/NSP hybrid vector pBY0BDa was created by four fragment ligation: the vector backbone from pBYR0-LBV1 digested with AgeI-SacI, the SIR-containing SacI-AvaI fragment from pBYR0-LBV1, the BDMV MP amplified from pBC1e with primers BC1-Xba-F and BC1-Ava-R (5′-CACCCGAGTTATTGCAACGATTTGGGCT, SEQ ID NO. 14) digested with XbaI-AvaI, and the BeYDV LIR amplified from pBY027 with primers LIR-C-Xba-R (5′-GGTCTAGAGTTGTTGTGACTCCGAGG, SEQ ID NO. 15) and M13RHT (5′-GGAAACAGCTATGACCATG, SEQ ID NO. 16) digested with AgeI-XbaI. Modifications to the promoters, 5′ UTRs, terminators, and other elements in pBY0BDa were carried out similarly and are shown in Table 1 as well as in the supplementary material. A T-DNA dual replicon vector pBYBDa-L5GFP (referred to as pBYBDx in Table 1, where “x” denotes the indicated letter) containing both the mastreviral GFP replicon and the BDMV MP/NSP hybrid replicon was created by four fragment ligation: the AscI-SacI backbone fragment and SacI-ApaI (GFP-TMV-LIR) fragment from pBYR-L5GFP were ligated with the ApaI-NsiI (LIR-MP) and NsiI-AscI (MP-SIR-NSP-LIR) fragments from pBY0BDx. For vector pBYBDf3.0 containing a 3.0 kb GFP replicon, the SacI-ApaI fragment from the ligation shown above was substituted with the SacI-ApaI fragment from pBYR2eFa-GFP (Diamos et al., 2016). For vector pBYBDf3.9 containing a 3.9 kb GFP replicon, the backbone of pBYBDf3.0 digested SbfI-HindIII was ligated with the SbfI-HindIII fragment containing the 35S promoter with duplicated enhancer and intron-containing extensin terminator from pBYR2e-GFP (Diamos et al., 2016).

The construction of the empty vector pPS1 was described previously in Huang and Mason, 2004.

An HA epitope tag was added to BDMV NSP by amplifying pBV1e with BV1-Xba-F and BV1HA-Sac-R (5′-tatGAGCTCTCATGCGTAATCTGGCACATCATAAGGATAACCGATATAA TCAAGGTCAAACG, SEQ ID NO. 17) designed to add HA tag to the 3′ end of NSP. The product was digested XbaI-SacI and cloned into pGFPe-TMV to yield pHA-BV1e. HA-tagged MP was generated similarly with the primer BC1HA-Sac-R (5′-tatgAGCTCTTATGCGTAATC TGGCACATCATAAGGATATTGCAACGATTTGGGCTG, SEQ ID NO. 18). Vectors containing MP and NSP driven by the NOS promoter were created by three fragment ligation: the vector backbone from pBI101 (Jefferson et al., 1987) digested with NheI-Sac, the NOS promoter amplified from pRep111 (Diamos and Mason 2018b) with primers PNOS-Xho-R (5′-GGCTCGAGTTTGGATTGAGAGTGAATATGAGAC, SEQ ID NO. 19) and PNOS-Asc-F (5′-acggcgcgccCATGAGCGGAGAATTAAGGG, SEQ ID NO. 20) digested with NheI-XhoI, and either the MP from pBC1e or NSP from pBV1e digested with XhoI-SacI, creating either pNOS-BC1 or pNOS-BV1.

AbMV NSP was amplified from pMDC7-HA-NSP (Kleinow et al., 2009) with primers AbBV1-Xba-F (5′-cgTCTAGAATGTACCCGTCTAGGAATAAA, SEQ ID NO. 21) and AbBV1-Sac-R (5′-agGAGCTCTTAACCAATATAGTCAAGGTCAAAC, SEQ ID NO. 22), digested with XbaI-SacI, and ligated into either pBV1e digested likewise to yield pABV1e, or into pBYR0-LBV1 digested likewise to create pBYR0-LABV1. AbMV MP was amplified from pMDC7-c-Myc-MP (Kleinow et al., 2009) with primers AbBC1-Bsr-F (5′-ggTGTACAATGGATTCTCAGTTAGTAAATCCTC, SEQ ID NO. 23) and AbBC1-Kpn-R (5′-gcGGTACCTTATTTCAATGATTTGGCTTGAGAAG, SEQ ID NO. 24), digested with BsrGI-KpnI, and ligated into pBD209 digested likewise to yield pBD209-AbBC1. The AbMV TMV 5′ UTR-AbMV MP-35S terminator fragment was excised from pBD209-AbBC1 by XhoI-EcoRI digestion and ligated with the backbone fragment from pBC1e digested with XhoI-ApaLI and the small ApaLI-EcoRI fragment from pBC1e to yield pABC1e. The hybrid vector pBY0ABa was created by four fragment ligation: the vector backbone from pBYR0-LABV1 digested with AgeI-SacI; the SIR-containing SacI-AvaI fragment from pBYR0-LABV1; the AbMV MP amplified from pBD209-ABC1 with primers AbBC1-Bsr-F and AbBC1-Ava-R (5′-gcCCCGAGTTATTTCAATGATTTGCTTGAGAAG, SEQ ID NO. 25) digested with BsrGI, blunted, then AvaI; and the BeYDV LIR amplified from pBY027 with primers LIR-C-Xba-R (5′-GGTCTAGAGTTGTTGTGACTCCGAGG, SEQ ID NO. 26) and M13RHT (5′-GGAAACAGCTATGACCATG, SEQ ID NO. 27) digested with XbaI, blunted, then digested with AgeI. pBY0ABa was used to create dual replicon hybrid vectors (pBYAMa and pBYAMb) with pBYR-L5GFP as described for pBY0BDa.

All genetic elements were sequence verified, as were any introduced mutations, any clones using segments amplified by PCR, and any clones generated with blunt ligations. Subclones made by transferring sequence-verified elements using standard restriction/ligation with compatible ends were verified by diagnostic PCR and diagnostic digests.

The specific constructs described herein listed below:
pBYR-LGFP (SEQ ID NO. 28): A GFP expression vector with GFP fused to the BeYDV LIR. Contains BeYDV Rep/RepA to allow replicon formation and amplification.
pBYR-V1GFP: Same as pBYR-LGFP except that BeYDV V1 is present in its native position driven by the LIR promoter, and GFP is fused downstream from V1.
pBYR-V1SGFP: Same as pBYR-V1GFP except that the intergenic region between V1 and V2 from wildtype BeYDV has been inserted between V1 and GFP.
pBYR-V1mutSGFP: Same as pBYR-V1SGFP except that the V1 start codon has been mutated.
pBYR-L5GFP: Same as pBYR-LGFP except that the tobacco mosaic virus 5′ UTR has been inserted between the LIR and GFP.
pHA-BV1e (35S-NSP): Produces HA-tagged BDMV NSP driven by the strong cauliflower mosaic virus 35S promoter and tobacco extensin 3′ UTR. Provides p19 RNA suppressor of RNA silencing.
pHA-BC1e (35S-MP): Like pHA-BV1e except NSP is replaced by MP.
pBV1e (35S-NSP): Same as pHA-BV1e except HA tag is removed.
pBC1e (35S-MP): Same as pHA-BC1e except HA tag is removed.
pNOS-BV1 (NOS-NSP): Contains BDMV NSP driven by the agrobacterium NOS promoter and NOS terminator.
pNOS-BC1 (NOS-MP): Contains BDMV MP driven by the agrobacterium NOS promoter and NOS terminator.
pPS1: Empty vector containing the 35S promoter and soybean vspB terminator with no gene insert.
pBYBDa (SEQ ID NO. 29): Contains two replicons which will be replicated separately in the plant nucleus after agrobacterium delivery: 1) a GFP replicon identical to pBYR-L5GFP; and 2) a replicon supplying BDMV NSP driven by the BeYDV v-sense LIR promoter with TMV 5′ UTR, and also supplying BDMV MP driven by the BeYDV c-sense LIR promoter. The bidirectional BeYDV SIR terminator resides between the MP and NSP. For a graphic depiction as well as the complete sequence of the MP/NSP replicon, see “S1.3 Construct Sequence Information.”
pBYBDb (SEQ ID NO. 30): Like pBYBDa except that the TMV 5′ UTR on NSP is removed.
pBYBDc (SEQ ID NO. 31): Like pBYBDb except that the potato pinII terminator is added to NSP, and the NSP start codon sequence context contains (GGAATG) mutation to reduce NSP expression.
pBYBDd (SEQ ID NO. 32): Like pBYBDb except the LIR v-sense promoter contains TATA-box mutation (TATAAG) designed to reduce expression of NSP.
pBYBDf (SEQ ID NO. 33): Like pBYBDb except that the soybean vspB 3′ UTR has been added to NSP.
pBYBDh (SEQ ID NO. 33): Like pBYBDf except that the position of the MP and NSP have been switched (LIR v-sense driving MP, LIR c-sense driving NSP).
pBYBDi (SEQ ID NO. 35): Like pBYBDb except that the tobacco extensin 3′ UTR has been added to NSP.
pBYBk (SEQ ID NO. 36): Like pBYBDb except that the tobacco etch virus 5′ UTR and 35S 3′ UTR has been added to NSP.
pBYB1 (SEQ ID NO. 37): Like pBYBDf except that the vspB 3′ UTR is placed downstream from MP instead of NSP.
pBYBDm (SEQ ID NO. 38): Like pBYBDb except that the potato pinII 3′ UTR has been added to NSP.
pBYBDp (SEQ ID NO. 39): Like pBYBDb except that the BDMV intergenic region that naturally occurs between MP and NSP has been placed between MP and NSP.
pBYBDq (SEQ ID NO. 40): Like pBYBDb except that the TMV 5′ UTR is placed upstream from MP.
pBYBDr (SEQ ID NO. 41): Like pBYBDc except that the TMV 5′ UTR is placed upstream from MP.
pBYAMa (SEQ ID NO. 42): Like pBYBDb except that the abutilon mosaic virus MP and NSP replacing the BDMV MP and NSP.
pBYAMb (SEQ ID NO. 43): Like pBYBDb except that the abutilon mosaic virus MP replaces the BDMV NSP.
pBYBDf3.0 (SEQ ID NO. 44): Contains an MP/NSP replicon identical to pBYBDf. The GFP replicon has been modified to include the 35S promoter driving GFP, increasing the replicon size.
pBYBDf3.9 (SEQ ID NO. 45): Like pBYBDf3.0 except that the 35S promoter contains duplicated enhancer regions, and the intron-containing tobacco extensin terminator has replaced the intronless terminator.

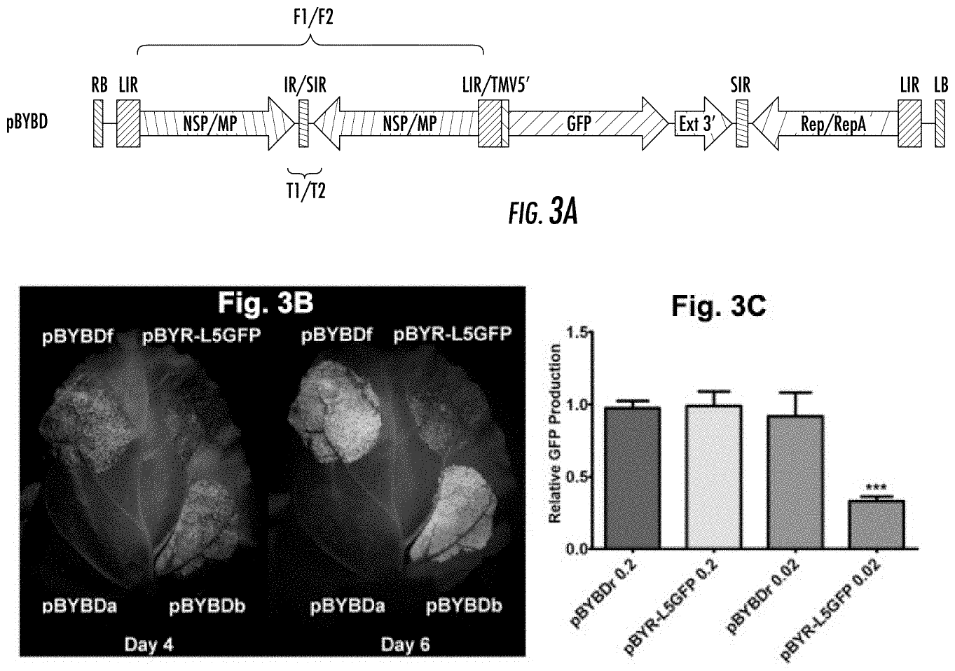

Table 1 describes the genetic components for expression of BDMV MP and NSP using the disclosed chimeric replicon, as shown in FIG. 3A. A GFP replicon vector identical to pBYR-L5GFP is also present in all constructs.

TABLE 1
Chimeric BeYDV/BDMV Dual Replicon Vector Components

| Vector | F1 | LIRv | T1 | SIR | T2 | LIRc | F2 | Size | Activity |
|---|---|---|---|---|---|---|---|---|---|
| BYBDa | TMV 5′ | NSP | None | SIR | — | MP | — | 2,247 | − |
| BYBDb | — | NSP | None | SIR | — | MP | — | 2,183 | ++ |
| BYBDc | LIR Mut1 | NSP | PinII | SIR | — | MP | — | 2,491 | ++ |
| BYBDd | LIR Mut2 | NSP | None | SIR | — | MP | — | 2,183 | + |
| BYBDf | — | NSP | VspB | SIR | — | MP | — | 2,733 | + |
| BYBDh | — | MP | VspB | SIR | — | NSP | — | 2,733 | −−− |
| BYBDi | — | NSP | Ext | SIR | — | MP | — | 2,658 | −−− |
| BYBDk | TEV 5′ | NSP | 35S | SIR | — | MP | — | 2,533 | −−− |
| BYBDl | — | NSP | None | SIR | VSP | MP | — | 2,748 | − |
| BYBDm | — | NSP | PinII | SIR | — | MP | — | 2,492 | + |
| BYBDp | — | NSP | None | BD | — | MP | — | 2,017 | + |
| BYBDq | — | NSP | None | SIR | — | MP | TMV 5′ | 2,243 | ++ |
| BYBDr | LIR Mut1 | NSP | PinII | SIR | — | MP | TMV 5′ | 2,562 | +++ |

F1, upstream modifications from MP/NSP driven by v-sense LIR promoter; LIRv, the gene driven by the v-sense LIR promoter; T1, additional terminator for LIRv in addition to SIR; SIR, bidirectional terminator region from either BeYDV (SIR) or BDMV (BD); T2, additional terminator for LIRc in addition to SIR; LIRc, gene driven by the c-sense LIR promoter; F2, upstream modifications from the gene driven by LIRc; TMV 5′, 5′ UTR from tobacco mosaic virus; LIR Mut1; the start codon sequence context contains (GGAATG) mutation to reduce gene expression, with mutation in bold; LIR Mut2; the LIR TATA-box contains (TATAAG) mutation to reduce gene expression; TEV 5′, 5′ UTR from tobacco etch virus; PinII, the potato proteinase inhibitor II terminator; VspB, the soybean vegetative storage protein B terminator; Ext, the tobacco intronless extensin terminator; 35S, the 35S terminator from cauliflower mosaic virus.

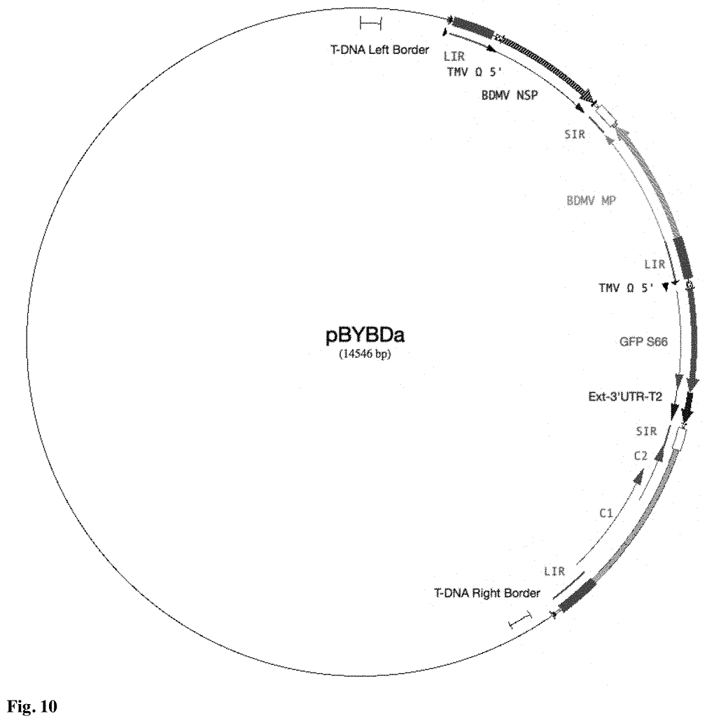

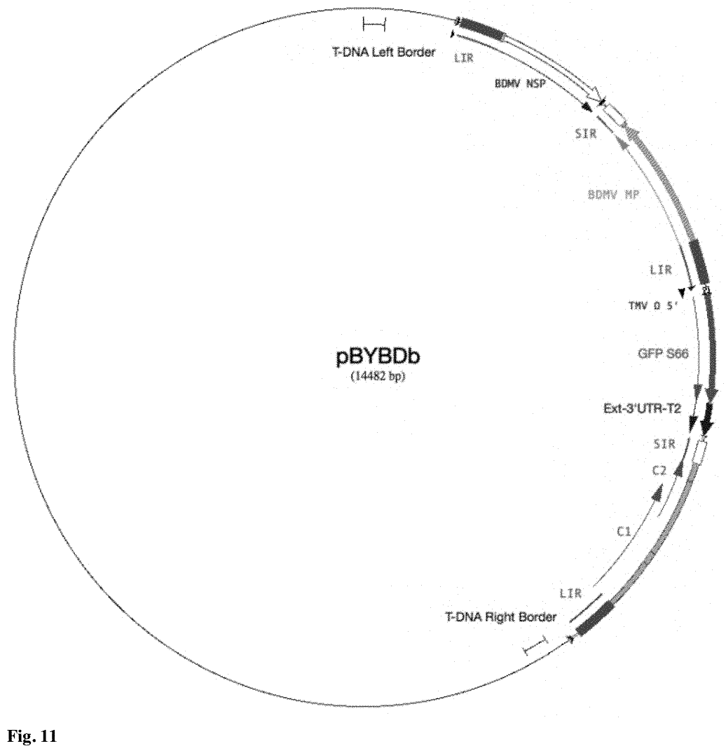

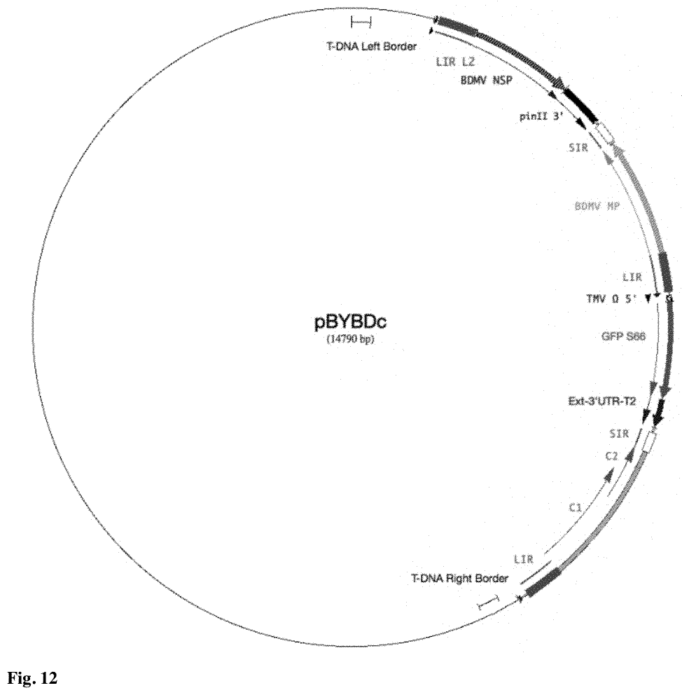

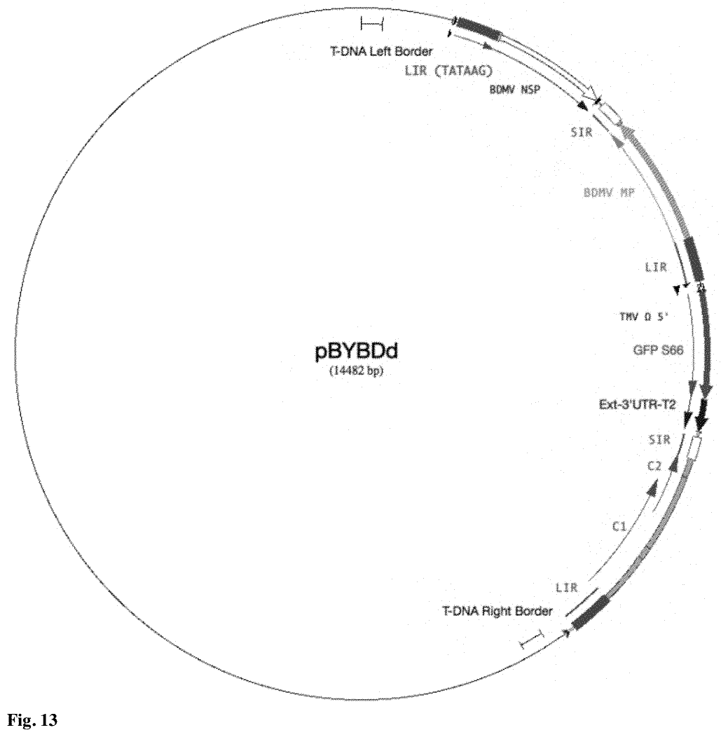

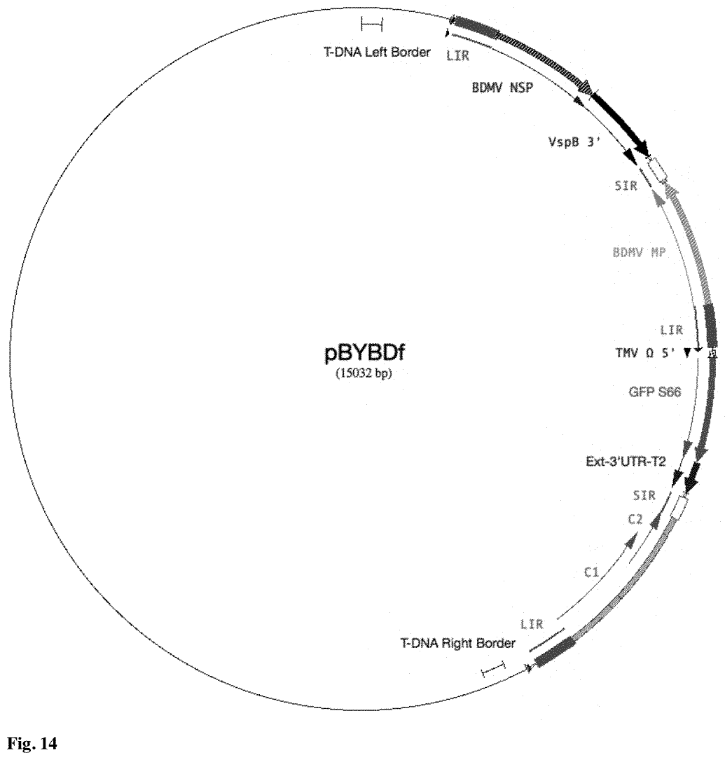

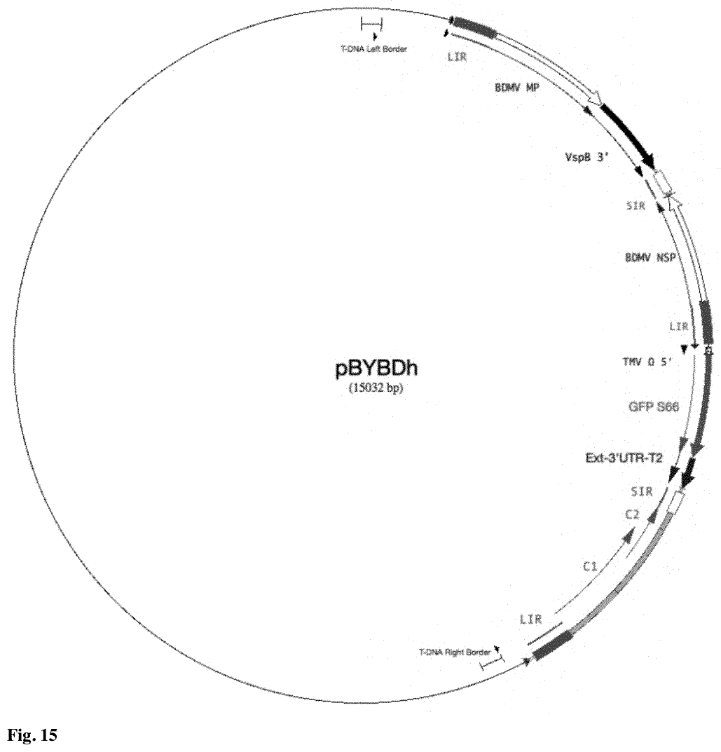

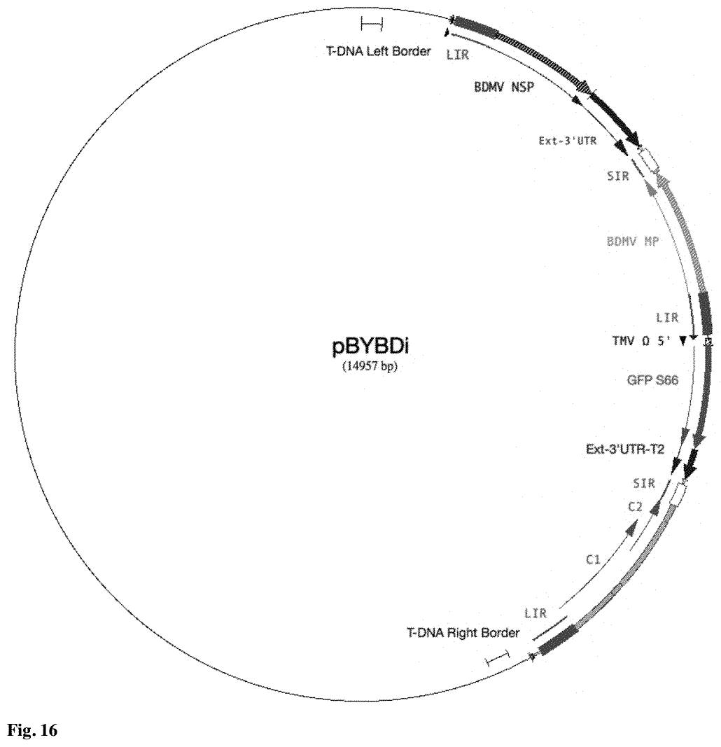

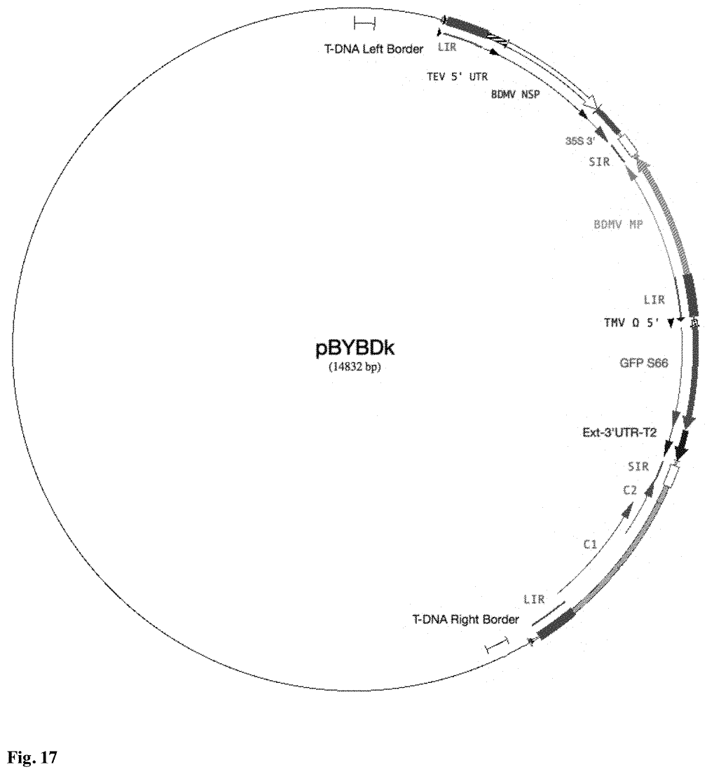

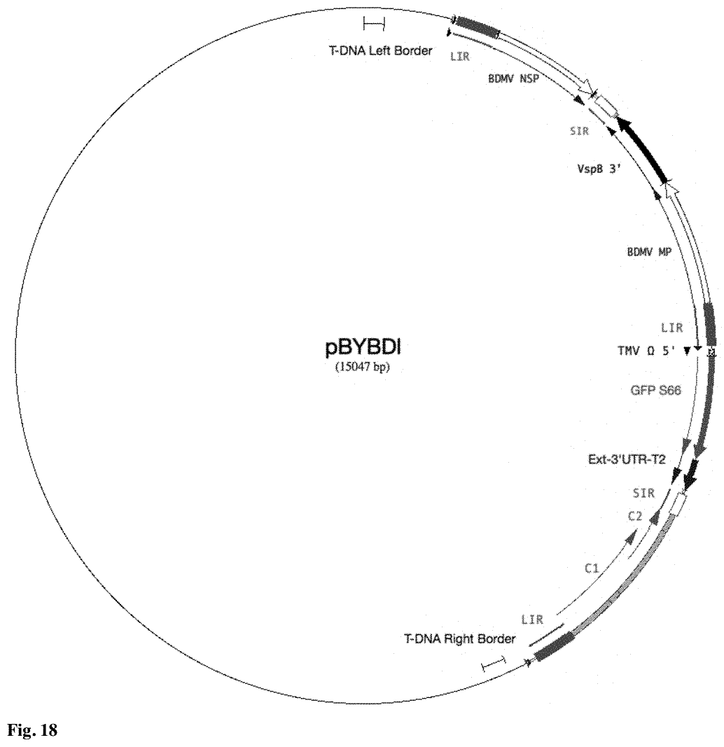

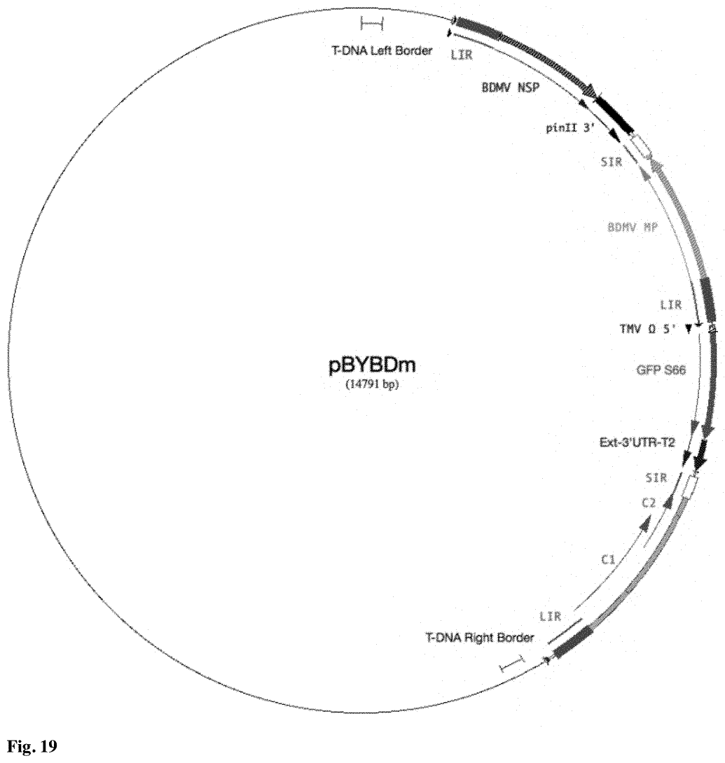

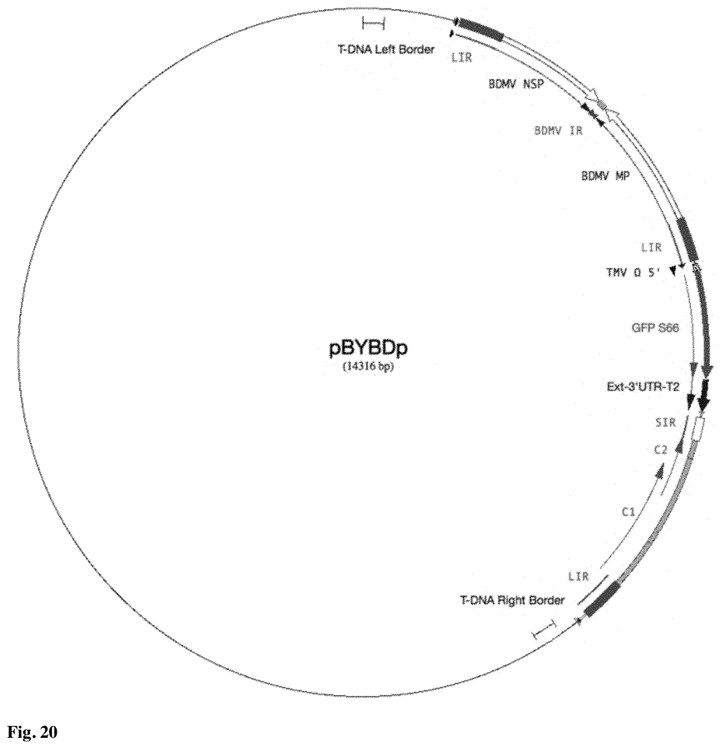

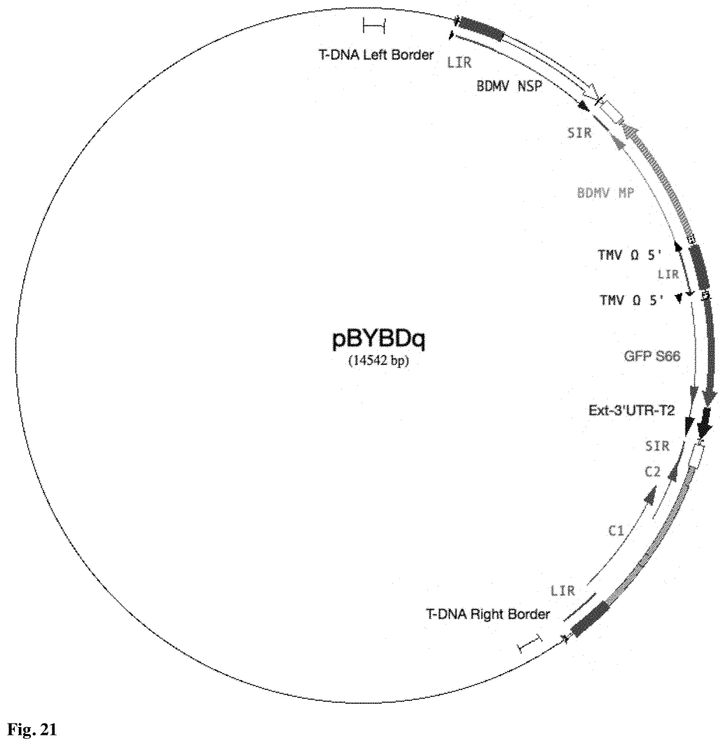

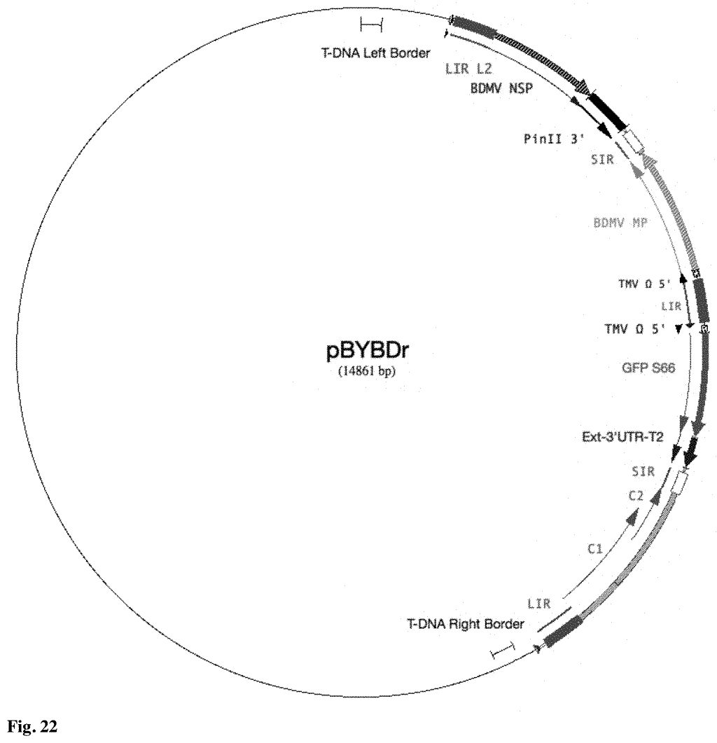

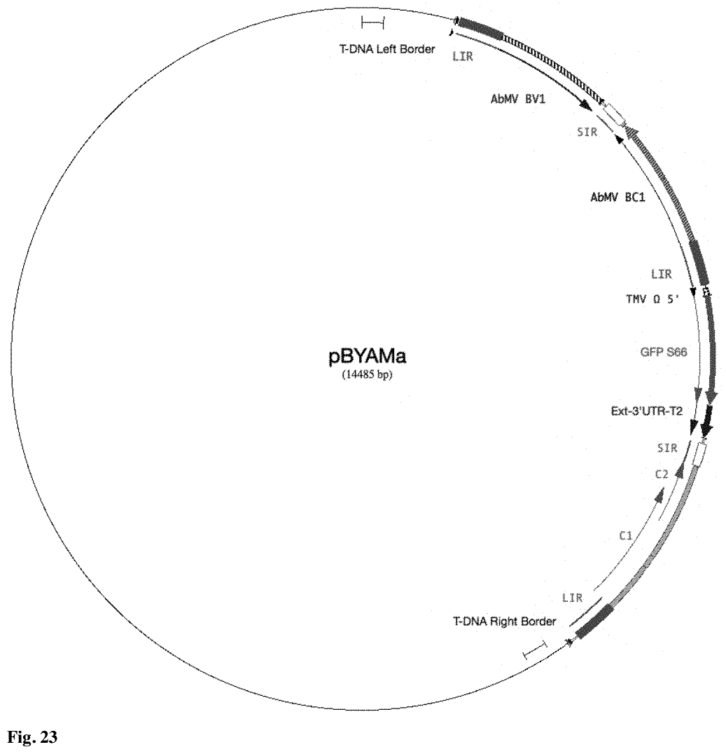

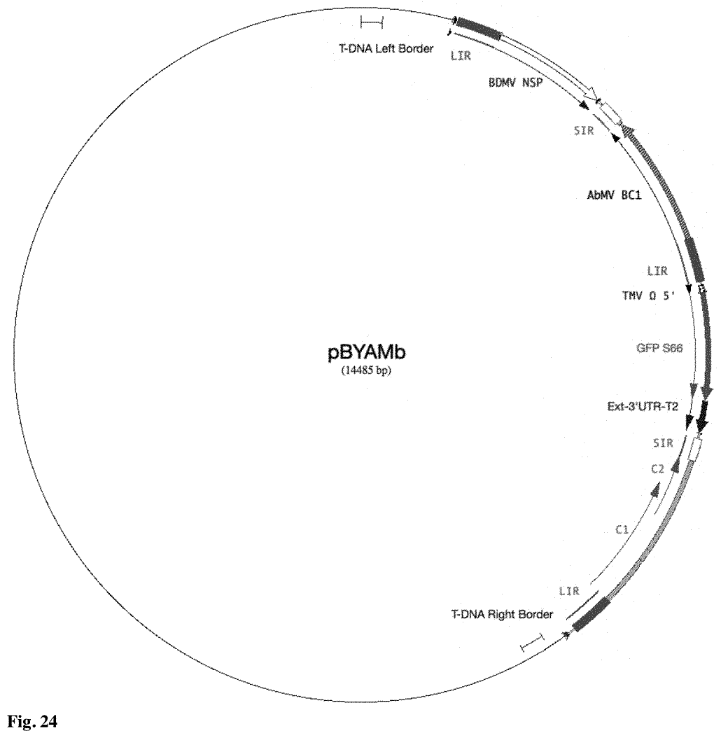

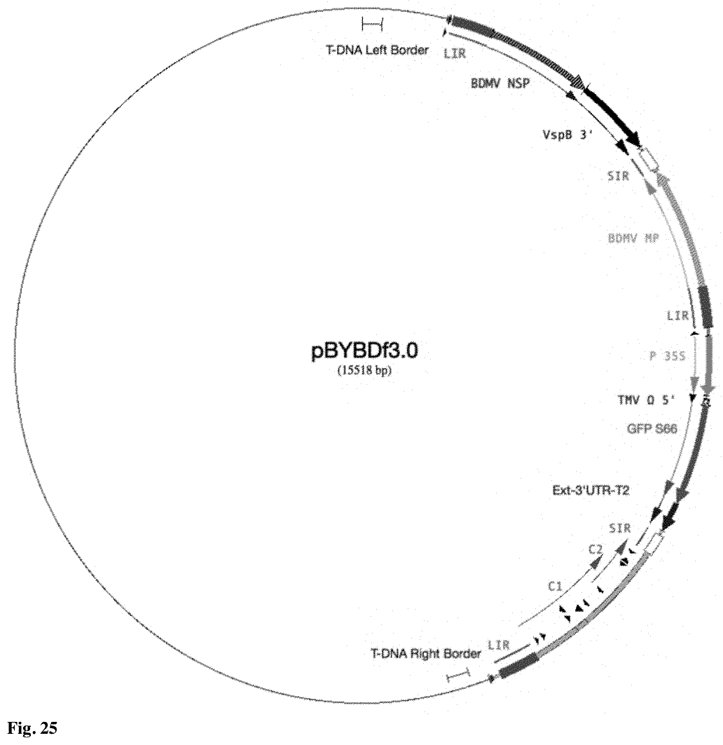

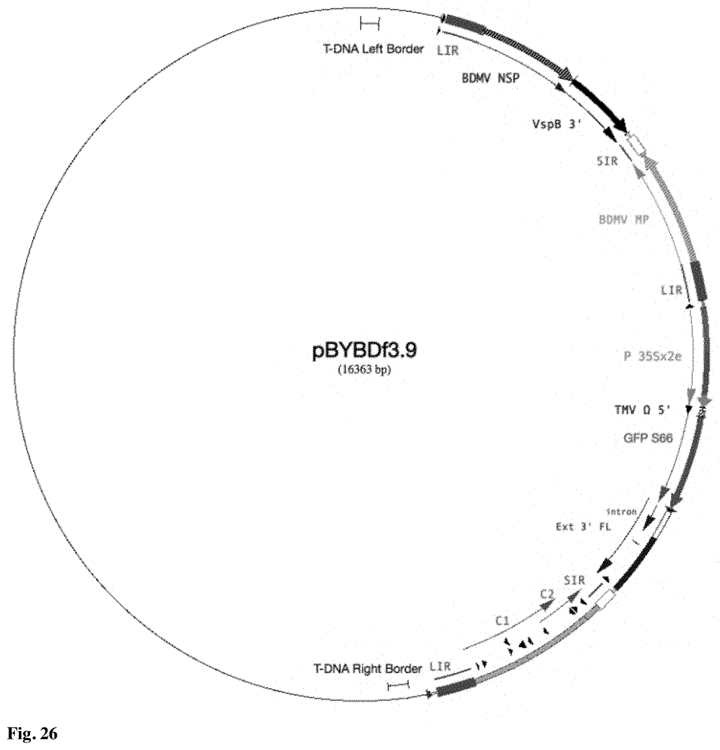

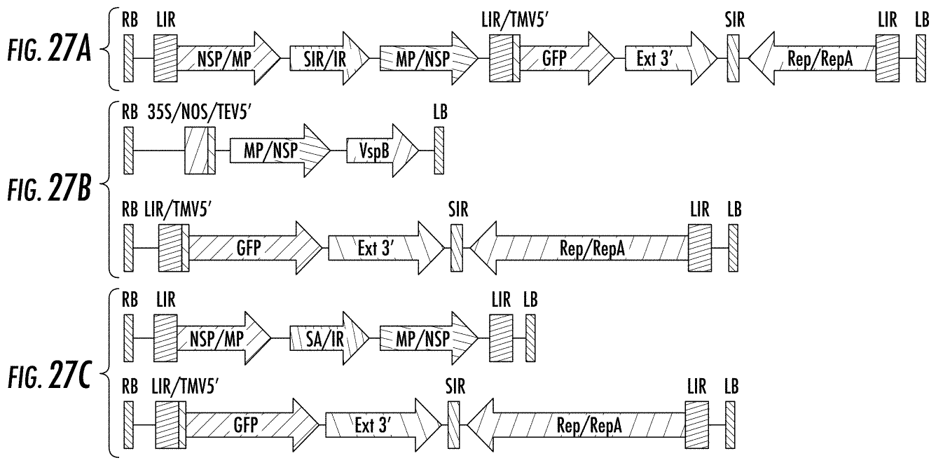

The plasmid maps for the GFP and MP/NSP replicon constructs is given in FIGS. 9-26 and the corresponding DNA sequence of the replicon contained in that vector are provided in below. For hybrid vectors containing two replicons, only the sequence of the MP/NSP replicon is given, as the GFP replicon is identical to pBYR-L5GFP. However, for size-increased GFP replicons, the GFP replicon sequence is instead given, as the MP/NSP replicon is identical to pBYBDf.

### 2. Construction of a Replication-Competent, Movement-Deficient Mastreviral Reporter System

To create a system to study geminivirus movement in Nicotiana benthamiana, a mastreviral GFP expression vector based on bean yellow dwarf virus (BeYDV) was constructed for delivery by agroinfiltration. GFP was fused to the BeYDV LIR virion-sense promoter which natively drives expression of the MP and CP.

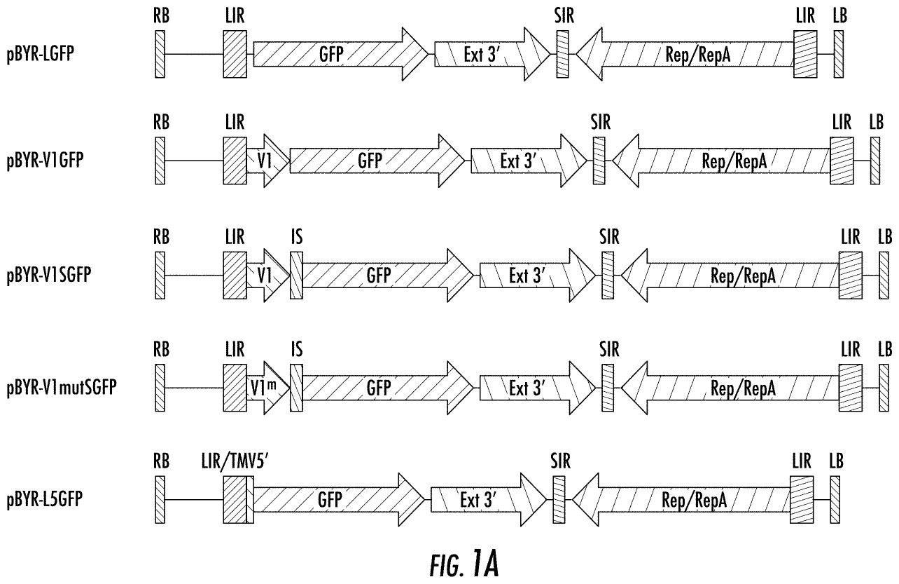

The vector was designed to contain the BeYDV rep and repA genes for vector replication, with the CP and/or MP replaced by GFP (FIG. 1A). Both sets of genes were placed under the control of the bidirectional LIR promoter, which was positioned on both ends of the vector to allow replicational release of the viral replicon in the plant nucleus after delivery by agrobacterium (Mor 2003 and Grimsley 1987). In the native configuration, the LIR promoter drives expression of both MP and CP, though the mechanism by which each is produced is not fully understood. Replacing the CP by fusing the complete GFP coding sequence directly downstream from the BeYDV MP coding sequence (construct pBYRV1-GFP) resulted in no detectable GFP expression (FIG. 1B). However, if a small native intergenic region between the movement and coat protein ORFs was preserved (pBYRV1S-GFP), GFP was produced at low levels (FIG. 1B), which indicates that this intergenic sequence is likely involved in CP expression.

While an intron was shown to be active in the MP gene of maize streak virus (Wright et al., 1997), none has been detected in BeYDV, though it has been suggested that the CP in BeYDV may be produced by translational frameshifting (Dekker et al., 1991). When the CP was replaced with GFP, MP with the mutated start codon prevented GFP expression. These data support a mechanism such as translational frameshifting, which would require initiation to first occur at MP for later CP translation (FIG. 1B).

To verify that MP translation is necessary for CP production, the MP start codon was mutated (pBYRV1 mS-GFP), which abolished GFP production despite the presence of an intact start codon in the GFP gene (FIG. 1B). These results suggest that CP is not produced by leaky scanning past the MP start codon, but instead requires both MP initiation and the intergenic sequence upstream of the CP for translation to occur. Replacement of both the movement and coat proteins by directly fusing GFP to the LIR in an optimal translation initiation context (pBYR-LGFP) resulted in substantially higher levels of GFP fluorescence (FIG. 1B). To further enhance the utility of this modified BeYDV vector as a reporter system, the tobacco mosaic virus 5′ UTR was inserted upstream from GFP. The resulting 2,587 bp vector (pBYR-L5GFP), which is very close in size to the authentic 2,561 bp genome of BeYDV, produced intense green fluorescence when infiltrated into plant leaves (FIG. 1B), and was found to replicate to a very high copy number, accumulating large quantities of replicon DNA which were visible on ethidium bromide stained gel (FIG. 1C), characteristic of robust mastreviral replication [29]. As the movement and coat proteins were removed, no cell-to-cell or systemic movement was detected.

Additionally, a small intervening sequence between the MP and CP coding sequences was required for GFP expression, though GFP expression was still low in this configuration. This suggests a role for the native CP coding sequence in efficient CP production. Fusion of GFP directly downstream from the LIR resulted in robust GFP production, indicating that the low levels of observed GFP expression with CP replacements likely did not result from a lack of transcriptional capacity by the LIR promoter, providing further support for translation control as the key determining factor in MP/CP production. When GFP was placed in an optimal translation initiation context (ACAAUG) (Sugio et al., 2010), especially with the TMV 5′ UTR, a known translational enhancer (Gallie, 2010), very high levels of GFP were produced from the LIR promoter (FIG. 1B).

3. Coexpression of BDMV MP and NSP with BeYDV GFP Replicons

Previous attempts to create a recombinant mastreviral system capable of cell-to-cell movement using the maize streak virus CP and MP were unsuccessful (Palmer et al., 2003). In agreement with these results, our experiments with the BeYDV MP and CP either from separate expression vectors, on BeYDV replicons, or from wildtype genomes all failed to achieve cell-to-cell movement of recombinant genomes. Instead, the potential for begomovirus movement proteins to interact with recombinant mastreviral genomes was investigated as they have been reported to interact with DNA molecules in a sequence nonspecific manner. The BDMV MP and NSP were each separately cloned with an HA epitope tag into an agrobacterium T-DNA binary vector under the control of the strong 35S promoter, tobacco etch virus 5′ UTR, and soybean vspB terminator. This vector was previously used to efficiently produce BeYDV Rep and RepA in Nicotiana benthamiana leaves (Huang et al., 2009). Each vector was separately agroinfiltrated into the leaves of Nicotiana benthamiana, and harvested leaf tissue was found to produce readily detectable MP and NSP (FIG. 2A). To ensure that the HA tag did not interfere with protein function, unmodified MP and NSP were also produced and used for all subsequent experiments.

To assess the interaction between BeYDV replicons and the BDMV MP and NSP, the replicating vector pBYR-L5GFP was compared with or without coinfiltration of 35S-MP and 35S-NSP. While the vector alone produced intense green fluorescence, GFP expression was strongly inhibited by addition of MP and NSP together (FIG. 2B). To determine whether the MP or NSP, or both, were responsible for this inhibition, each gene was infiltrated separately with the replicating GFP vector. While coinfiltration of 35S-MP had no substantial effect on GFP expression, 35S-NSP strongly inhibited GFP expression (FIG. 2C). As the NSP has been shown to interact with genome-sized DNA molecules, and 35S-NSP produced high levels of NSP, overproduction of the NSP was hypothesized to sequester or otherwise interfere with BeYDV replicon utilization and prevent GFP production. Consistent with this hypothesis, coexpression of pBYR-L5GFP with 35S-NSP strongly inhibited replicon accumulation (FIG. 7A) and coexpression of the native BeYDV CP with pBYR-L5GFP, which has nuclear shuttling functions, also similarly inhibited GFP expression. Therefore, expression vectors were constructed with the much weaker nopaline synthase (NOS) promoter from agrobacterium to reduce production of NSP and/or MP. Unlike with 35S-MP/NSP, separate infiltration of either NOS-MP or NOS-NSP alone with the BeYDV vector had no detectable effect on GFP production (FIG. 2D). While infiltration of 35S-MP and 35S-NSP alone caused leaf chlorosis that progressed to necrotic lesions for NSP, NOS-MP and NOS-NSP produced no negative leaf symptoms (FIG. 2E, FIG. 7B). To test whether MP/NSP would cause spreading of GFP replicons, BeYDV GFP replicons were delivered to only a fraction of all the leaf cells using a low agrobacterium OD₆₀₀ of 0.002 while MP/NSP vectors were infiltrated with a much higher agrobacterium OD₆₀₀ of 0.1 to ensure they were supplied to most cells. While coinfiltration of either NOS-MP or NOS-NSP separately with pBYR-L5GFP had no notable effects (FIG. 2D), coinfiltration of both NOS-MP and NOS-NSP simultaneously with the replicating vector resulted in a detectable increase in GFP fluorescence in the infiltrated area by 5 DPI (FIG. 2F).

BDMV NSP strongly inhibited GFP replication and expression from genome-sized BeYDV replicons when produced from the strong 35S promoter (FIGS. 2B, 2C, and 7A), suggesting that NSP efficiently interacts with mastreviral replicons. High levels of NSP may reduce the availability of transcription templates, either by transport out of the nucleus or by interfering with replication, as NSP binds most DNA forms at high concentrations, forming collapsed structures (Hehnle et al., 2004; Rojas et al., 1998).

Increased cell death was also observed with overexpression of NSP (FIGS. 2E and 7B); however GFP inhibition occurred many days prior to the development of leaf necrosis. Specifically, both MP and NSP produced leaf chlorosis when expressed from the strong 35S promoter (FIG. 2E), and NSP leaf spots eventually developed necrotic lesions (FIGS. 7A and 7B). Expression of NSP from a weaker promoter did not appreciably impact GFP expression (FIG. 2D). Expression from the weaker NOS promoter produced no visible symptom development with either protein (FIG. 2E). Replicating vectors expressing MP and NSP at levels that produced efficient cell-to-cell movement had no notable differences in leaf toxicity compared to pBYR-L5GFP alone, nor did native BDMV DNA-B coinfiltrated with pBDA-GFP. However, replicating hybrid vectors overexpressing NSP produced higher levels of tissue necrosis.

In support of the general sequence-nonspecific transport capabilities of the BDMV MP and NSP to move genome-sized DNA molecules, coexpression of each by the NOS promoter caused an increase in GFP fluorescent area produced by recombinant mastreviral replicons (FIG. 2F).

### 4. Creation of Chimeric BeYDV/BDMV Dual Replicon Vectors

In native begomovirus infection, MP/NSP are expressed from replicating DNAs (DNA B), which, along with a rep-supplying replicon (DNA A), spread together from cell-to-cell. To more authentically model native replicon movement, a chimeric BeYDV/BDMV vector was created which expresses BDMV MP and NSP from the BeYDV bidirectional LIR promoter. The native BeYDV replication proteins were replaced by the BDMV MP, and the BeYDV coat and movement proteins were replaced by the BDMV CP. Importantly, while this vector is incapable of replicating on its own, it contains all of the necessary cis-elements for replication, and thus is capable of replication when Rep and RepA are supplied in trans from a replication-competent BeYDV vector (Mor et al., 2003). The chimeric BeYDV/BDMV genome uses the replication and transcription elements from BeYDV to produce MP and NSP from BeYDV replicons.

Geminiviruses contain bidirectional terminator elements at the ends of the c-sense and v-sense genes. For BeYDV and BDMV DNA-B, efficient termination requires the upstream coding sequence of the gene be terminated, the SIR in the case of BeYDV, and also the downstream reverse coding sequence of the opposite-sense gene (Diamos and Mason, 2018a). Geminivirus gene products are temporally regulated: in the early stages of infection, complementary-sense nonstructural gene products predominate, which then activate virion-sense gene products late in infection (Muñoz-Martin et al., 2003). Native NSP production requires transactivation late in infection by AC2 (Berger and Sunter, 2013), so NSP was inserted under the control of the late-acting virion-sense LIR promoter, which is similarly controlled by transactivation by Rep/RepA to mimic the native configuration of DNA-B.

To optimize expression of MP and NSP, and to modify the size of the vector, various vector configurations were constructed with different relative positions of the MP and NSP, and with modified 5′ UTR, terminator, and promoter elements (Table 1). Each of these modified hybrid vectors were then combined with the replication-competent GFP vector pBYR-L5GFP to create a single vector containing both replicons placed in tandem and separated by a LIR (FIG. 3A). This arrangement allows simultaneous delivery of both a Rep/GFP-containing replicon and a MP/NSP-containing replicon on a single agrobacterium T-DNA, which are individually released and replicated in a noncompeting manner in the plant nucleus (Huang 2010).

Hybrid BDMV/BeYDV vectors were compared to the original vector pBYR-L5GFP, which lacks MP/NSP, at a low agrobacterium OD₆₀₀ of 0.002 to allow monitoring of individual GFP foci for movement. Numerous MP/NSP vector iterations strongly inhibited GFP expression, likely due to improper expression levels of MP/NSP. By contrast, several vector configurations produced GFP spots which strongly increased in fluorescent size between 4-8 DPI, whereas GFP spots infiltrated with pBYR-L5GFP reached peak fluorescence around 4 DPI and remained unchanged thereafter (FIG. 3B). To quantitatively assess GFP expression by these vectors, protein was extracted from agroinfiltrated leaf material and analyzed by SDS-PAGE. When hybrid or MP/NSP deficient vectors were assayed for GFP expression at an agrobacterium OD₆₀₀ of 0.2, which is sufficient to deliver GFP replicons to most plant cells, no differences in GFP expression were observed (FIG. 3C). By contrast, GFP expression by the hybrid vector pBYBDr was 3-fold greater at an agrobacterium OD₆₀₀ of 0.02 (FIG. 3C). Different configurations designed to alter the relative expression levels of MP and NSP in the BeYDV replicon strongly impacted vector performance (FIG. 3D, Table 1).

The hybrid BDMV/BeYDV vectors provided very high levels of GFP expression even when the agrobacterium concentration was reduced by a factor of 10, unlike vectors lacking the BDMV MP and NSP (FIG. 3C). Attempts at reversing the positions of MP and NSP strongly inhibited GFP expression, which could be the result of rapid accumulation of NSP (FIG. 3D, Table 1). Furthermore, efficient cell-to-cell movement was highly sensitive to the precise levels of MP and NSP being produced. Increasing NSP expression by addition of a stronger 5′ UTR or additional terminator, which has previously shown to be a general enhancer of gene expression (Beyene et al., 2011; Diamos et al., 2016; Diamos and Mason, 2018a), severely inhibited GFP replicon expression. However, supplying insufficient NSP also strongly limited function.

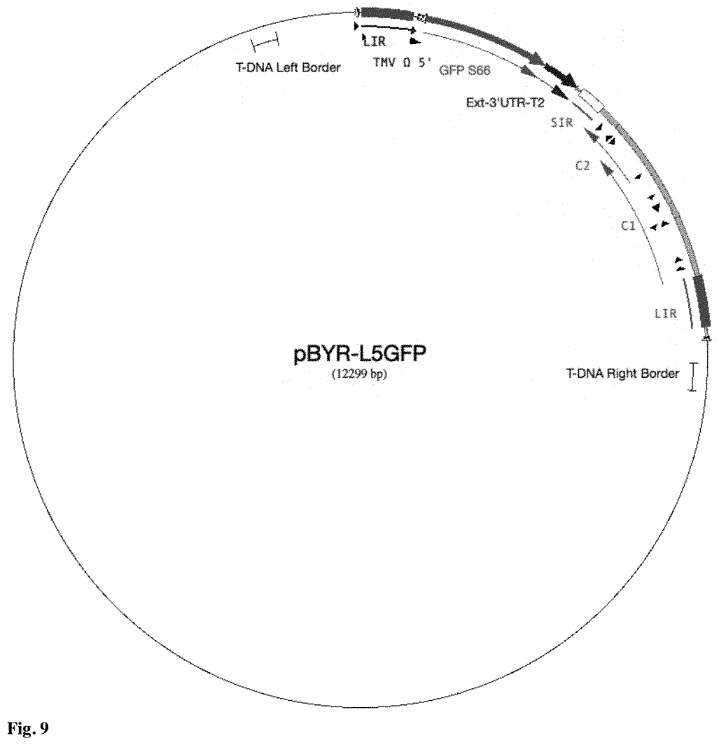

Mutation of the LIR v-sense TATA-box (TATAAA to TATAAG) decreased promoter activity by an estimated 50% without impacting replication (FIGS. 8A and 8B). While the hybrid vector pBYBDb-L5GFP produced efficient GFP spreading, introduction of the v-sense TATA-box mutation to reduce NSP expression also reduced GFP fluorescent area by 65% (FIG. 3D).

When the native 3′ ends of BDMV DNA-B, including the wildtype intergenic region, were used in the chimeric vector pBYBDp, GFP fluorescent area was strongly reduced (FIG. 3D). Because the BeYDV LIR was the only difference between this vector and the BDMV DNA-B, and because DNA-B supplied levels of MP and NSP adequate for efficient movement (FIG. 3D/4B), intrinsic differences in promoter activity between BDMV and BeYDV may produce improper levels of MP and/or NSP. The small replicon size of this vector (2,017 bp) also could have affected its performance, though the 2,183 bp MP/NSP replicon of pBYBDb was highly functional.

### 5. BDMV MP and NSP Provide Cell-to-Cell Movement to Mastreviral Replicons

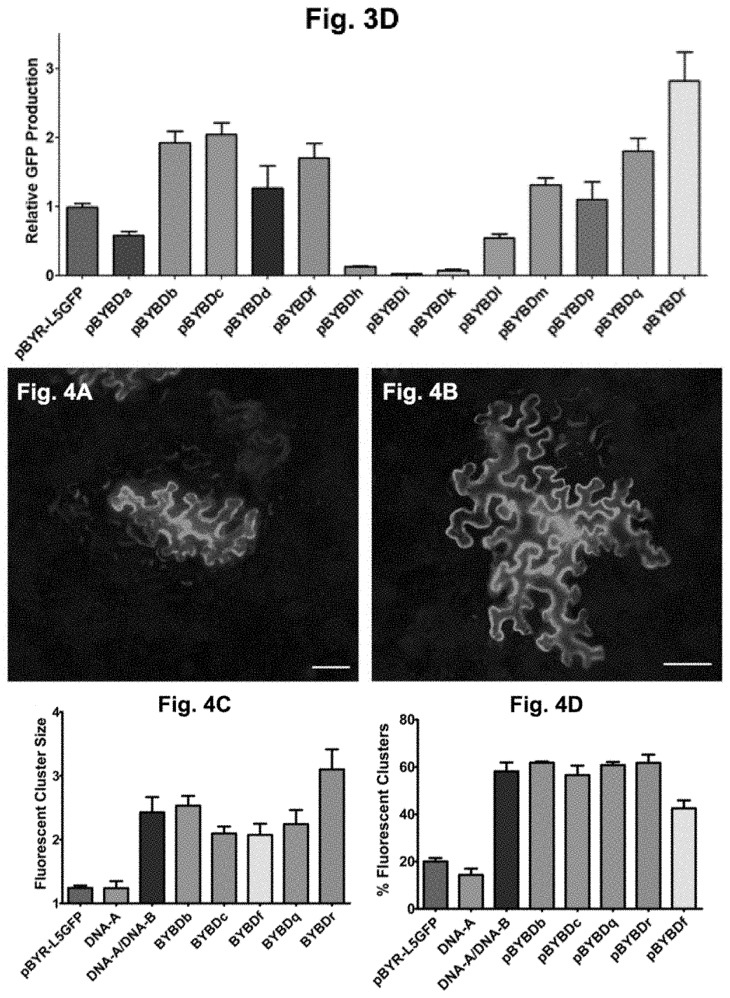

To further study the cell-to-cell movement of hybrid BDMV/BeYDV vectors, fluorescence microscopy of agroinfiltrated leaf tissue was performed. Leaves of Nicotiana benthamiana were agroinfiltrated with either hybrid BeYDV/BDMV vectors containing MP/NSP, or pBYR-L5GFP lacking MP/NSP, using a low agrobacterium OD₆₀₀ of 0.0002 to produce predominately isolated infected cells. As a control for authentic geminiviral movement, the T-DNA vector pBDA-GFP, which contains BDMV DNA-A modified to produce GFP in place of the coat protein (Sudarshana 1998), was agroinfiltrated either alone or in combination with pBDB, a T-DNA vector containing BDMV DNA-B to supply MP and NSP in their native configuration. Leaf spots agroinfiltrated with only pBDA-GFP contained mostly isolated fluorescent cells, with an average fluorescent cell cluster size of 1.2±0.1 cells at 6 DPI. By contrast, leaf spots coinfiltrated with pBDA-GFP and pBDB produced predominately clusters of fluorescent cells, with an average fluorescent cluster size of 2.4±0.2 (FIG. 4A/B). Both constructs produced similar levels of intense fluorescence in individual leaf cell cytoplasm and nuclei (FIG. 4A). Similar to the results observed with BDMV DNA-AB supplying vectors, leaf spots agroinfiltrated with pBYR-L5GFP alone showed mostly isolated fluorescent cells, whereas leaf spots infiltrated with chimeric BDMV/BeYDV vectors were found to contain larger aggregates of fluorescent cells similar in size to those observed for native BDMV (FIG. 4A/B). The average cluster size of fluorescent cells in the hybrid vector pBYBDr was 3.1±0.3 compared to 1.2±0.04 for pBYR-L5GFP. Interestingly, vector configurations differed both in the extent of movement, and the proportion of cells showing movement. The larger 2,733 bp replicon from pBYBDf produced fewer cells displaying cell-to-cell movement, and a reduced number of adjacent fluorescing cells compared to pBYBDr (FIG. 4C/4C). Together, these results indicate that the BDMV MP and NSP allow cell-to-cell spread of mastreviral replicons at efficiencies similar to native begomoviral replicons when the expression levels of MP and NSP are optimized.

By fluorescence microscopy, MP and NSP expressing replicons produced fluorescent cell clusters similar in size to those reported for BDMV replicons (Gilbertson et al., 2003; Levy and Czosnek, 2003), which agreed with our results coinfiltrating BDMV DNA-B and DNA-A (FIG. 4C). While geminivirus NSP has been shown to be involved in preventing host translation shut-off (Zorzatto et al., 2015), there was no difference in individual cell fluorescence (FIG. 4A). Only in the number of fluorescent cells differed (FIG. 4C), which correlated with the increased GFP fluorescent area observed in leaves and the measured GFP expression data (FIG. 3B/D). Additionally, increased GFP expression required that replicons were not delivered to all cells (FIG. 3C), and no increased expression or evidence of movement was seen when MP or NSP were delivered separately (FIG. 2).

### 6. BeYDV and BDMV Replicon Size Restricts Movement

The BDMV MP and NSP have been reported to bind DNA in a size-dependent manner. To test whether chimeric BeYDV/BDMV vectors were size-restricted, a dual-replicon vector (pBYBDf-3.9) was constructed which contained an unmodified MP/NSP replicon as well as a substantially larger 3.9 kb GFP replicon. The larger replicon was constructed by insertion of a 35S promoter driving GFP, as well as insertion of a longer form of the extensin 3′ UTR. These modifications were designed to increase replicon size while minimally altering the activity of the vector, as the LIR and 35S promoters are both strong, and the short and long forms of the extensin terminator have not been found to vary substantially in activity in BeYDV vectors (Rosenthal et al., 2018).

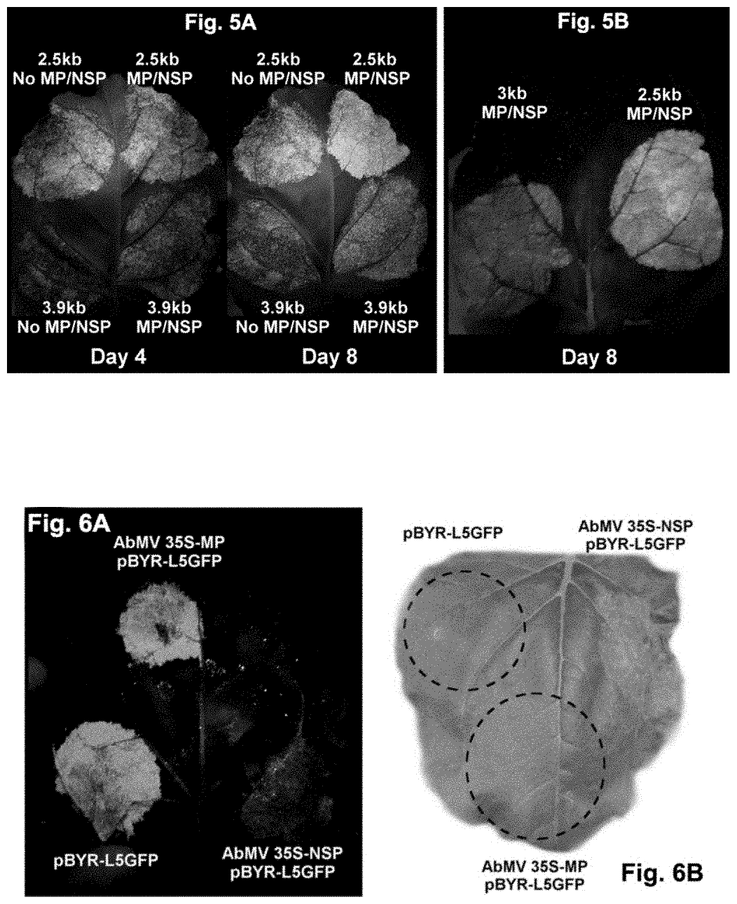

Using size-increased mastrevirus replicons, movement function by MP and NSP was inhibited by even a small increase from a 2.5 kb to a 3.0 kb replicon, and severely inhibited with a 3.9 kb replicon (FIG. 5A/B). While the original vector containing a 2.5 kb GFP replicon was found to substantially increase in GFP fluorescence between 4 and 8 DPI, the vector containing the larger 3.9 kb replicon had only minimal changes in GFP fluorescence after 4 DPI (FIG. 5A). A 3.1 kb vector containing an intermediately sized 3′ UTR also displayed inhibited GFP fluorescence after 4 DPI compared to the 2.5 kb vector (FIG. 5B), in agreement with previous findings that the BDMV MP/NSP function primarily with genome-sized DNA molecules. Gilbertson et al. reported that MP-mediated movement of 3.4 kb DNA molecules was moderately limited and 5.5 kb DNA molecules were severely limited in Nicotiana benthamiana leaves, likely due to intrinsic limitations imposed by endogenous cell-to-cell trafficking through the plasmodesmata (Gilbertson et al., 2003). However, pBYBDb, which contains a 2,183 bp MP/NSP replicon, had strong evidence of movement (FIG. 3D/4B), indicating that geminiviral replicons smaller than the native genome size are also functional.

Efficient GFP spreading required genome-size replicons (FIGS. 5A and 5B). Functional movement was observed only with 2.1-2.7 kb replicons (FIGS. 3D and 4B). NSP may interact with large replicons, as high levels of NSP inhibit replication and expression from replicons as large as 4 kb (data not shown), in agreement with the hypothesis that MP, not NSP, is responsible for the strict replicon size limitations.

Taken together, these data strongly suggest that the observed increase in GFP fluorescence results from cell-to-cell movement, not from an increase in GFP expression in individual cells or from diffusion of GFP between cells.

7. Characterization of AbMV MP and NSP Expression with BeYDV Replicons

The AbMV MP and NSP have shown similar DNA-binding properties to BDMV (Hehnle et al., 2004). AbMV is restricted to the phloem, whereas the related BDMV can spread through epidermal, cortical and phloem cells in Phaseolus vulgaris (Wang et al., 1996; Wege et al., 2000). Attempts to overexpress AbMV MP and NSP in transgenic plants have failed due to the toxic effects of the proteins, however successful expression was achieved using a transient inducible system (Kleinow et al., 2009). Prior studies have found that complementing the DNA-A from AbMV with the DNA-B of BDMV conferred enhanced tissue invasion to the AbMV DNA-A, successfully moving AbMV out of the phloem (Levy and Czosnek, 2003). Interestingly, the DNA-B from AbMV did not limit the movement capacity of BDMV DNA-A, despite its phloem limitation when paired with its own DNA-A. Additionally, a begomovirus lacking DNA-B entirely was complemented in movement function by both a curtovirus and a topocuvirus mixed infection (Briddon and Markham, 2001).

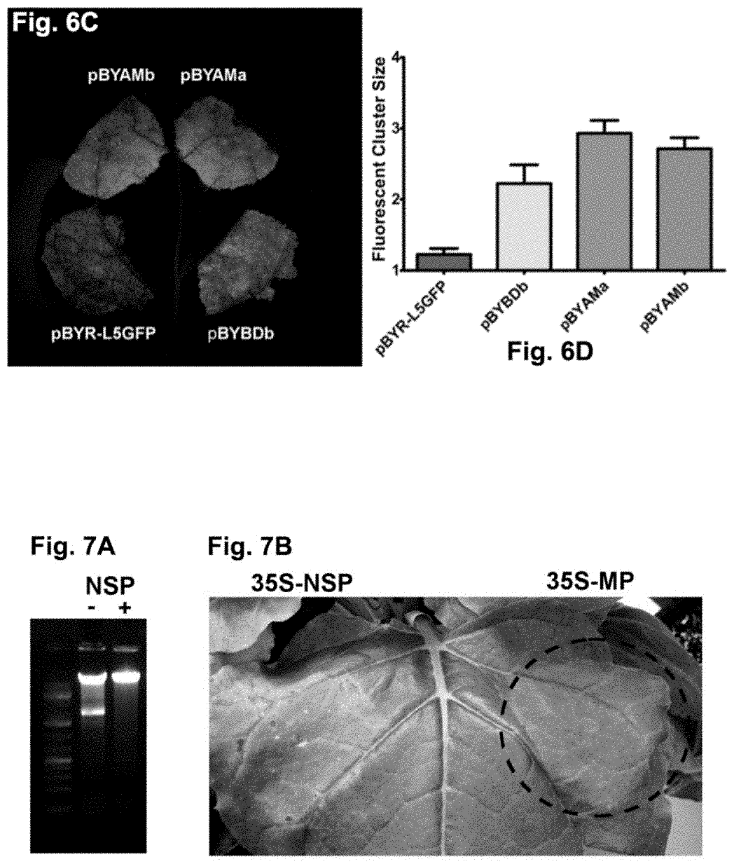

To explore whether AbMV MP and NSP can also function with mastreviral replicons in planta, AbMV MP and NSP was produced in Nicotiana benthamiana leaves. Despite the phloem limitation of the native AbMV, AbMV NSP interacts with BeYDV replicons similarly to BDMV (FIG. 6A). Similar to the results found for BDMV NSP, overexpression of AbMV NSP strongly inhibited expression from BeYDV GFP replicons (FIG. 6A) whereas MP did not. When expressed at high levels, AbMV NSP also elicited the plant hypersensitive response, whereas MP only produced leaf chlorosis (FIG. 6B).

Chimeric AbMV/BeYDV dual replicon vectors modeled after functional BDMV/BeYDV vectors were constructed and tested for their capacity to provide functional cell-to-cell movement. Similar to the results observed with BDMV, significant increases in leaf fluorescent area were observed with the AbMV MP and NSP (vector pBYAMa) when agroinfiltrated at low concentrations, which corresponded to increased numbers of adjacent fluorescent cells (FIGS. 6C and 6D). In fact, AbMV NSP and MP can move BeYDV replicons cell-to-cell with similar or greater efficiencies compared to BDMV MP and NSP when expressed at appropriate levels (FIGS. 6C and 6D). A chimeric AbMV/BDMV/BeYDV replicon expressing the BDMV NSP, the AbMV MP, and the BeYDV replication machinery (vector pBYAMb) was also created, and the chimeric replicon can robustly replicate, express high levels of GFP, and efficiently move cell-to-cell in Nicotiana benthamiana leaves (FIGS. 6C and 6D).

These data also demonstrate that the phloem limitation of AbVM does not arise from the intrinsic function of the MP and NSP themselves but rather due to insufficient MP and NSP expression by AbMV in non-phloem cells.

### 8. Methods

a. Agroinfiltration of Nicotiana benthamiana Leaves

Binary vectors were separately introduced into Agrobacterium tumefaciens EHA105 by electroporation. The resulting strains were verified by restriction digestion or PCR, grown overnight at 30° C., and used to infiltrate leaves of 5- to 6-week-old Nicotiana benthamiana maintained at 23-25° C. Briefly, the bacteria were pelleted by centrifugation for 5 min at 5,000 g and then resuspended in infiltration buffer (10 mM 2-(N-morpholino)ethanesulfonic acid (MES), pH 5.5 and 10 mM MgSO₄) to the OD₆₀₀ indicated in each experiment. The resulting bacterial suspensions were injected by using a syringe without needle into leaves through a small puncture (Huang and Mason, 2004). Plant tissue was harvested between 4-8 days post infiltration (DPI) as indicated in each experiment. Leaves producing GFP were photographed under UV illumination generated by a B-100AP lamp (UVP, Upland, Calif.). GFP fluorescent cells were observed with a Zeiss Axio Scope A1.

b. Protein Extraction, SDS-PAGE, and Western Blot

Total protein extract was obtained by homogenizing agroinfiltrated leaf samples with 1:5 (w:v) ice cold extraction buffer (25 mM sodium phosphate, pH 7.4, 100 mM NaCl, 1 mM EDTA, 0.1% Triton X-100, 10 mg/mL sodium ascorbate, 0.3 mg/mL PMSF) using a Bullet Blender machine (Next Advance, Averill Park, N.Y.) following the manufacturer's instruction. The crude plant extract was clarified by centrifugation at 13,000 g for 10 min at 4° C.

Clarified plant protein extract was mixed with sample buffer (50 mM Tris-HCl, pH 6.8, 2% SDS, 10% glycerol, 0.02% bromophenol blue) and separated on 4-15% polyacrylamide gels (Bio-Rad, Hercules, Calif., USA). For GFP fluorescence, PAGE gels were visualized under UV illumination (365 nm). PAGE gels were stained with Coomassie stain (Bio-Rad) following the manufacturer's instructions. The 26 kDa band corresponding to GFP was analyzed using ImageJ software to quantify the band intensity (Gassmann et al., 2009). For western blot, polyacrylamide gels were transferred to PVDF membranes, blocked with 5% dry milk in PBST (PBS with 0.05% tween-20) for 1 h at 37° C. and probed in succession with rabbit anti-HA (1:2000) and goat anti-rabbit IgG-horseradish peroxidase conjugated (Sigma-Aldrich, St. Louis, Mo.) diluted 1:10,000 in 1% PBSTM. Bound antibody was detected with ECL reagent (Amersham, Little Chalfont, United Kingdom).

c. Plant DNA Extraction and Replicon Visualization

Agroinfiltrated leaf samples (100 mg) were harvested and ground to a fine powder in liquid nitrogen. Total DNA was extracted from ground leaf samples using the DNeasy Plant Mini Kit (Qiagen, Hilden, Germany) according to the manufacturer's instructions. DNA (1 μg) was separated on 1% agarose gels stained with ethidium bromide and visualized under UV light to observe replicon production.

### REFERENCES CITED

Azzam O, Frazer J, de la Rosa D, Beaver J S, Ahlquist P, et al. Whitefly transmission and efficient ssDNA accumulation of bean golden mosaic geminivirus require functional coat protein. Virology 1994; 204:289-96.
Berger M R, Sunter G. Identification of sequences required for AL2-mediated activation of the tomato golden mosaic virus-yellow vein BR1 promoter. J Gen Virol 2013; 94:1398-1406.
Beyene G, Buenrostro-Nava M T, Damaj M B, Gao S-J, Molina J, et al. Unprecedented enhancement of transient gene expression from minimal cassettes using a double terminator. Plant Cell Rep 2011; 30:13-25.
Briddon R W, Markham P G. Complementation of bipartite begomovirus movement functions by topocuviruses and curtoviruses. Arch Virol 2001; 146:1811-1819.
Briddon R W, Pinner M S, Stanley J, Markham P G. Geminivirus coat protein gene replacement alters insect specificity. Virology 1990; 177:85-94.
Comai L, Moran P, Maslyar D. Novel and useful properties of a chimeric plant promoter combining CaMV 35S and MAS elements. Plant Mol Biol 1990; 15:373-381.
Dekker E L, Woolston C J, Xue Y B, Cox B, Mullineaux P M. Transcript mapping reveals different expression strategies for the bicistronic RNAs of the geminivirus wheat dwarf virus. Nucleic Acids Res 1991; 19:4075-81.
Diamos A G, Mason H S. Chimeric 3′ flanking regions strongly enhance gene expression in plants. Plant Biotechnol J 2018; 16:1971-1982 (Diamos 2018a).
Diamos A G, Mason H S. Modifying the Replication of Geminiviral Vectors Reduces Cell Death and Enhances Expression of Biopharmaceutical Proteins in Nicotiana benthamiana Leaves. Front Plant Sci 2018; 9:1974 (Diamos 2018b).
Diamos A G, Rosenthal S H, Mason H S. 5′ and 3′ Untranslated Regions Strongly Enhance Performance of Geminiviral Replicons in Nicotiana benthamiana Leaves. Front Plant Sci 2016; 7:200.
Duffy S, Shackelton L A, Holmes E C. Rates of evolutionary change in viruses: patterns and determinants. Nat Rev Genet 2008; 9:267-276.
Gallie D R. The 5′-leader of tobacco mosaic virus promotes translation through enhanced recruitment of eIF4F. Nucleic Acids Res 2002; 30:3401-3411.
Garcia-Andrés S, Monci F, Navas-Castillo J, Moriones E. Begomovirus genetic diversity in the native plant reservoir Solanum nigrum: evidence for the presence of a new virus species of recombinant nature. Virology 2006; 350:433-442.
Gassmann M, Grenacher B, Rohde B, Vogel J. Quantifying Western blots: pitfalls of densitometry. Electrophoresis 2009; 30:1845-55.
Gilbertson R L, Sudarshana M, Jiang H, Rojas M R, Lucas W J. Limitations on Geminivirus Genome Size Imposed by Plasmodesmata and Virus-Encoded Movement Protein: Insights into DNA Trafficking. Plant Cell 2003; 15:2578-2591.
Grimsley N, Hohn T, Davies J W, Hohn B. Agrobacterium-mediated delivery of infectious maize streak virus into maize plants. Nature 1987; 325:177-179.
Hanley-Bowdoin L, Bejarano E R, Robertson D, Mansoor S. Geminiviruses: masters at redirecting and reprogramming plant processes. Nat Rev Microbiol 2013; 11:777-88.
Hehnle S, Wege C, Jeske H. Interaction of DNA with the movement proteins of geminiviruses revisited. J Virol 2004; 78:7698-7706.
Hou Y-M, Paplomatas E J, Gilbertson R L. Host Adaptation and Replication Properties of Two Bipartite Geminiviruses and Their Pseudorecombinants. Mol Plant-Microbe Interact MPMI 1998; 11:208-217.
Huang Z, Chen Q, Hj elm B, Arntzen C, Mason H. A DNA replicon system for rapid high-level production of virus-like particles in plants. Biotechnol Bioeng 2009; 103:706-714.
Huang Z, Mason H S. Conformational analysis of hepatitis B surface antigen fusions in an Agrobacterium-mediated transient expression system. Plant Biotechnol J 2004; 2:241-9.
Huang Z, Phoolcharoen W, Lai H, Piensook K, Cardineau G, et al. High-level rapid production of full-size monoclonal antibodies in plants by a single-vector DNA replicon system. Biotechnol Bioeng 2010; 106:n/a-n/a.
Hussain M, Mansoor S, Tram S, Fatima A N, Zafar Y. The nuclear shuttle protein of Tomato leaf curl New Delhi virus is a pathogenicity determinant. J Virol 2005; 79:4434-9.
Jefferson R A, Kavanagh T A, Bevan M W. GUS fusions: beta-glucuronidase as a sensitive and versatile gene fusion marker in higher plants. EMBO J 1987; 6:3901-7.
Jeffrey J L, Pooma W, Petty I T. Genetic requirements for local and systemic movement of tomato golden mosaic virus in infected plants. Virology 1996; 223:208-18.
Jeske H. Geminiviruses. Current Topics in Microbiology and Immunology 2009; 331:185-226.
Kleinow T, Tanwir F, Kocher C, Krenz B, Wege C, et al. Expression dynamics and ultrastructural localization of epitope-tagged Abutilon mosaic virus nuclear shuttle and movement proteins in Nicotiana benthamiana cells. Virology 2009; 391:212-220.
Kraberger S, Harkins G W, Kumari S G, Thomas J E, Schwinghamer M W, et al. Evidence that dicot-infecting mastreviruses are particularly prone to inter-species recombination and have likely been circulating in Australia for longer than in Africa and the Middle East. Virology 2013; 444:282-291.
Krenz B, Jeske H, Kleinow T. The induction of stromule formation by a plant DNA-virus in epidermal leaf tissues suggests a novel intra- and intercellular macromolecular trafficking route. Front Plant Sci 2012; 3:291.
Krenz B, Windeisen V, Wege C, Jeske H, Kleinow T. A plastid-targeted heat shock cognate 70 kDa protein interacts with the Abutilon mosaic virus movement protein. Virology 2010; 401:6-17.
Krichevsky A, Kozlovsky S V., Gafni Y, Citovsky V. Nuclear import and export of plant virus proteins and genomes. Mol Plant Pathol 2006; 7:131-146.
Lefeuvre P, Moriones E. Recombination as a motor of host switches and virus emergence: Geminiviruses as case studies. Curr Opin Virol 2015; 10:14-19.
Legg J P, Fauquet C M. Cassava mosaic geminiviruses in Africa. Plant Mol Biol 2004; 56:585-599.
Levy A, Czosnek H. The DNA-B of the non-phloem-limited bean dwarf mosaic virus (BDMV) is able to move the phloem-limited Abutilon mosaic virus (AbMV) out of the phloem, but DNA-B of AbMV is unable to confine BDMV to the phloem. Plant Mol Biol 2003; 53:789-803.
Liu H, Boulton M I, Thomas C L, Prior D A, Oparka K J, et al. Maize streak virus coat protein is karyophyllic and facilitates nuclear transport of viral DNA. Mol Plant Microbe Interact 1999; 12:894-900.
Liu L, Pinner M S, Davies J W, Stanley J. Adaptation of the geminivirus bean yellow dwarf virus to dicotyledonous hosts involves both virion-sense and complementary-sense genes. J Gen Virol 1999; 80:501-506.
Lucas W J. Plant viral movement proteins: Agents for cell-to-cell trafficking of viral genomes.

Virology 2006; 344:169-184.
Monjane A L, Martin D P, Lakay F, Muhire B M, Pande D, et al. Extensive Recombination-Induced Disruption of Genetic Interactions Is Highly Deleterious but Can Be Partially Reversed by Small Numbers of Secondary Recombination Events. J Virol 2014; 88:7843-7851.
Monjane A L, Pande D, Lakay F, Shepherd D N, van der Walt E, et al. Adaptive evolution by recombination is not associated with increased mutation rates in Maize streak virus. BMC Evol Biol 2012; 12:252.
Mor T S, Moon Y-S, Palmer K E, Mason H S. Geminivirus vectors for high-level expression of foreign proteins in plant cells. Biotechnol Bioeng 2003; 81:430-437.
Muñoz-Martin A, Collin S, Herreros E, Mullineaux P M, Fernández-Lobato M, et al. Regulation of MSV and WDV virion-sense promoters by WDV nonstructural proteins: A role for their retinoblastoma protein-binding motifs. Virology 2003; 306:313-323.
Palmer K E, Rybicki E P. Investigation of the potential of Maize streak virus to act as an infectious gene vector in maize plants. Arch Virol 2001; 146:1089-1104.
Richter L J, Thanavala Y, Arntzen C J, Mason H S. Production of hepatitis B surface antigen in transgenic plants for oral immunization. Nat Biotechnol 2000; 18:1167-71.
Rojas M R, Hagen C, Lucas W J, Gilbertson R L. Exploiting Chinks in the Plant's Armor: Evolution and Emergence of Geminiviruses. Annu Rev Phytopathol 2005; 43:361-394.
Rojas M R, Noueiry A O, Lucas W J, Gilbertson R L. Bean dwarf mosaic geminivirus movement proteins recognize DNA in a form- and size-specific manner. Cell 1998; 95:105-113.
Rosenthal S H, Diamos A G, Mason H S. An intronless form of the tobacco extensin gene terminator strongly enhances transient gene expression in plant leaves. Plant Mol Biol 2018; 96:429-443.
Scholthof K B G, Adkins S, Czosnek H, Palukaitis P, Jacquot E, et al. Top 10 plant viruses in molecular plant pathology. Mol Plant Pathol 2011; 12:938-954.
Shen W H, Hohn B. Vectors based on maize streak virus can replicate to high copy numbers in maize plants. J Gen Virol 1995; 76:965-969.
Shepherd D N, Mangwende T, Martin D P, Bezuidenhout M, Kloppers F J, et al. Maize streak virus-resistant transgenic maize: A first for Africa. Plant Biotechnol J 2007; 5:759-767.
Sudarshana M R, Wang H L, Lucas W J, Gilbertson R L. Dynamics of Bean Dwarf Mosaic Geminivirus Cell-to-Cell and Long-Distance Movement in Phaseolus vulgaris Revealed, Using the Green Fluorescent Protein. Mol Plant-Microbe Interact 1998; 11:277-291.
Sugio T, Matsuura H, Matsui T, Matsunaga M, Nosho T, et al. Effect of the sequence context of the AUG initiation codon on the rate of translation in dicotyledonous and monocotyledonous plant cells. J Biosci Bioeng 2010; 109:170-173.
van der Walt E, Palmer K E, Martin D P, Rybicki E P. Viable chimaeric viruses confirm the biological importance of sequence specific maize streak virus movement protein and coat protein interactions. Virol J 2008; 5:61.
Varsani A, Shepherd D N, Monjane A L, Owor B E, Erdmann J B, et al. Recombination, decreased host specificity and increased mobility may have driven the emergence of maize streak virus as an agricultural pathogen. J Gen Virol 2008; 89:2063-2074.
Waigmann E, Ueki S, Trutnyeva K, Citovsky V. The Ins and Outs of Nondestructive Cell-to-Cell and Systemic Movement of Plant Viruses. CRC Crit Rev Plant Sci 2004; 23:195-250.
Wang H L, Gilbertson R L, Lucas W J. Spatial and temporal distribution of Bean Dwarf Mosaic Geminivirus in <i>Phaseolu vulgaris<i/> and <i>Nicotiana benthamiana<i/>. Phytopathology 1996; 86:1204-1214.
Wege C, Gotthardt R D, Frischmuth T, Jeske H. Fulfilling Koch's postulates for Abutilon mosaic virus. Arch Virol 2000; 145:2217-2225.
Wright E A, Heckel T, Groenendijk J, Davies J W, Boulton M I. Splicing features in maize streak virus virion- and complementary-sense gene expression. Plant J 1997; 12:1285-1297.
Zhou Y-CY-C, Garrido-Ramirez E R, Sudarshana M R, Yendluri S, Gilbertson R L. The N-terminus of the Begomovirus nuclear shuttle protein (BV1) determines virulence or avirulence in Phaseolus vulgaris. Mol Plant Microbe Interact 2007; 20:1523-1534.
Zhou Y, Rojas M R, Park M-R, Seo Y-S, Lucas W J, et al. Histone H3 Interacts and Colocalizes with the Nuclear Shuttle Protein and the Movement Protein of a Geminivirus. J Virol 2011; 85:11821-11832.
Zorzatto C, Machado J P B, Lopes K V G, Nascimento K J T, Pereira W A, et al. NIK1-mediated translation suppression functions as a plant antiviral immunity mechanism. Nature 2015; 520:679-682.

We claim:

1. A T-DNA binary vector comprising a chimeric T-DNA region, wherein the T-DNA region comprises:
two replicons;
a first long intergenic region from bean yellow dwarf virus (BeYDV);
a second long intergenic region from BeYDV; and
a third long intergenic region from BeYDV,
wherein:
the first replicon is between the first and the second long intergenic regions from BeYDV,
the second replicon is an expression cassette and is between the second and the third long intergenic regions from BeYDV,
the first replicon comprises:
a first sequence encoding a begomovirus movement protein;
a second sequence encoding a begomovirus nuclear shuttle protein; and
a first short intergenic region from BeYDV, wherein the first short intergenic region separates the first and the second sequences, and
the second replicon comprises a third sequence encoding a transgene.

2. The T-DINA binary vector of claim 1, wherein:
the first sequence and the second sequence are both from bean dwarf mosaic virus (BDMV),
the first sequence and the second sequence are both from abutilon mosaic virus (AbMV), or
the first sequence is from AbMV and the second sequence is from BDMV.

3. The T-DNA binary vector of claim 1, wherein the second replicon further comprises a second short intergenic region from BeYDV and a fourth sequence encoding Rep/RepA from BeYDV, wherein the second short intergenic region separate the third and fourth sequences and wherein the fourth sequence is downstream of the third sequence.

4. The T-DNA binary vector of claim 1, wherein the second replicon further comprises 5′ UTR from tobacco mosaic virus (TMV 5′), wherein the TMV 5′ is upstream of the third sequence.

5. The T-DNA binary vector of claim 1, wherein the second replicon further comprises an intronless form of the gene terminator from tobacco extension (Ext 3′), wherein the Ext 3′ is downstream of the third sequence.

6. The T-DNA binary vector of claim 1, wherein the TATA box of at least one of the first LIR, the second LIR, and the third LIR is mutated and comprises the nucleic acid sequence TATAAG.

7. The T-DNA binary vector of claim 1, wherein the first replicon further comprises a truncated pinII terminator downstream of the second sequence encoding the begomovirus nuclear shuttle protein, wherein:
the first long intergenic region from BeYDV is a v-sense LIR comprising a mutated TATA box having the sequence TATAAC and is upstream of the second sequence encoding the begomovirus nuclear shuttle protein, and
the second long intergenic region from BeYDV is a c-sense LIR and is upstream of the first sequence encoding the begomovirus movement protein.

8. A method for expressing a recombinant protein in a plant cell, the method comprising administering to a plant cell a composition comprising an agrobacterium transformed with the T-DNA binary vector of claim 1, wherein the amount of agrobacterium administered as determined by OD₆₀₀ is less than 0.2.

9. A plant expression system facilitating cell-to-cell movement of a transgene comprising:
a first expression vector with a T-DNA region comprising a replicon spanning a first long intergenic region from bean yellow dwarf virus (BeYDV) and a second long intergenic region from BeYDV, wherein the replicon comprises:
a first sequence encoding a transgene;
a 5′ untranslated region (UTR) upstream of the first sequence;
a terminator downstream of the first sequence;
a second sequence encoding Rep/RepA from BeYDV; and
a short intergenic region from BeYDV,
wherein:
the first sequence, the 5′ UTR, and the terminator are upstream of the short intergenic region, and
the second sequence is downstream of the short intergenic region;
a second expression vector with a T-DNA region comprising:
a promoter selected from the group consisting of: cauliflower mosaic virus 35S promoter and agrobacterium NOS promoter;
a third sequence encoding a begomovirus movement protein; and
a terminator comprising a terminator selected from the group consisting of: agrobacterium NOS terminator and a truncated form of the gene terminator from tobacco extension (Ext 3′); and
a third expression vector with a T-DNA region comprising:
a promoter selected from the group consisting of: cauliflower mosaic virus 35S promoter and agrobacterium NOS promoter;
a fourth sequence encoding a begomovirus nuclear shuttle protein; and
a terminator selected from the group consisting of: agrobacterium NOS terminator and Ext 3′.

10. The plant expression system of claim 9, wherein the 5′ UTR in the first expression vector comprises 5′ UTR from tobacco mosaic virus.

11. The plant expression system of claim 9, wherein the terminator in the first expression vector comprises Ext 3′.

12. The plant expression system of claim 9, wherein the begomovirus movement protein and the begomovirus nuclear shuttle protein are from BDMV and/or AbMV.

13. The plant expression system of claim 9, wherein the TATA box of the third and/or the fourth LIR is mutated and comprises the nucleic acid sequence TATAAG.

14. A method for expressing a recombinant protein in a plant cell, the method comprising administering to a plant cell a composition comprising an agrobacterium transformed with the plant expression system of claim 9, wherein the amount of agrobacterium administered as determined by OD₆₀₀ is less than 0.2.

15. A plant expression system facilitating cell-to-cell movement of a transgene comprising:
a first expression vector with a T-DNA region comprising a first replicon spanning a first long intergenic region from bean yellow dwarf virus (BeYDV) and a second long intergenic region from BeYDV, the first replicon comprising:
a first sequence encoding a transgene;
a second sequence encoding Rep/RepA or one protein selected from the group consisting of: begomovirus movement protein and a begomovirus nuclear shuttle protein;
a short intergenic region from BeYDV,
wherein the short intergenic region from BeYDV separates the first sequence, and the second sequence; and
a second expression vector with a T-DNA region comprising a second replicon spanning a third long intergenic regions from BeYDV and a fourth long intergenic region from BeYDV, the second replicon comprising:
a third sequence encoding a protein selected from the group comprising: begomovirus movement protein and a begomovirus nuclear shuttle protein;
wherein the second sequence and the third sequence encode different proteins.

16. The plant expression system of claim 15, wherein the second sequence encodes one protein selected from the group consisting of: begomovirus movement protein and a begomovirus nuclear shuttle protein.

17. The plant expression system of claim 15, wherein the second sequence encodes Rep/RepA, the second replicon further comprises:
a fourth sequence encoding a protein selected from the group comprising: begomovirus movement protein and a begomovirus nuclear shuttle protein; and
a second short intergenic region from BeYDV,
wherein:
the third sequence and the fourth sequence encode different proteins, and
the second short intergenic region separates the third sequence and the fourth sequence.

18. The plant expression system of claim 15, wherein:
the begomovirus movement protein and the begomovirus nuclear shuttle protein are both from bean dwarf mosaic virus (BDMV),
the begomovirus movement protein and the begomovirus nuclear shuttle protein are both from abutilon mosaic virus (AbMV), or
the begomovirus movement protein is from AbMV and the begomovirus nuclear shuttle protein is from BDMV.

19. The plant expression system of claim 17, wherein the TATA box of the third and/or of the fourth LIR is mutated and comprises the nucleic acid sequence TATAAG.

20. A method for expressing a recombinant protein in plant cell, the method comprising administering to a plant cell a composition comprising an agrobacterium transformed with the plant expression system of claim 15, wherein the amount of agrobacterium administered as determined by OD₆₀₀ is less than 0.2.
